(19) United States
(12) Patent Application Publication
(10) Pub. No.: US 2025/0101488 A1
(43) Pub. Date: Mar. 27, 2025

(54) RAPID DIAGNOSTIC DRY REAGENT TEST FOR ANTIBIOTIC RESISTANCE, AND METHODS OF USE AND METHODS OF MAKING THEREOF

(71) Applicant: BioAmp Diagnostics, Inc., San Mateo, CA (US)

(72) Inventors: Tara Renee DeBoer, San Mateo, CA (US); Angel Resendez, San Mateo, CA (US)

(21) Appl. No.: 18/730,594

(22) PCT Filed: Jan. 27, 2023
(86) PCT No.: PCT/US2023/011762

§ 371 Date: Jul. 19, 2024

Related U.S. Application Data
(60) Provisional application No. 63/304,335, filed on Jan. 28, 2022.

(51) Int. Cl. C12Q 1/34; G01N 21/31

(52) U.S. Cl. CPC C12Q 1/34; G01N 21/31; G01N 2333/986

(57) ABSTRACT

Provided herein are reagent tests that can be used to identify specific types of antibiotic resistant microorganisms in a sample, and methods of use thereof.

### CROSS REFERENCE TO RELATED APPLICATIONS

This application claims priority under 35 U.S.C. § 119 from Provisional Application Ser. No. 63/304,335, filed Jan. 28, 2022, the disclosures of which are incorporated herein by reference.

### TECHNICAL FIELD

Provided herein are tests methods, systems and kits that can be used to identify specific types of antibiotic resistant microorganisms in a sample.

### BACKGROUND

Urinary tract infections (UTIs) remain one of the most common bacterial infections worldwide, and they are becoming increasingly resistant to many first-line antibiotics typically prescribed to treat these infections. Multidrug resistant bacteria were once limited to the walls of hospitals and now are rampant in the communities. Recent clinical outcome studies have reported >85% of patients seeking care for cUTIs found to be caused by a multidrug resistant organisms presented from the community and not from a long-term health or skilled nursing facility. Perhaps even more striking is the occurrence of multidrug resistant UTIs in infant and children under the age of 5. In general, diagnostic tests that can enable evidence-based treatment of UTIs are urgently needed to circumvent the selective pressure being applied by current empiric treatment practices that have given rise to highly drug-resistant bacterial pathogens. Therefore, diagnostics tests that can be performed across a wide range of healthcare settings are needed to address to the emerging needs of patients and healthcare systems witnessing increased impact of UTIs. Diagnostic tests that can provide clinical information required to direct care of UTIs must be (1) capable of fitting a wide range of workflows, (2) affordable, and (3) and able to be performed by unskilled personnel.

### SUMMARY

The disclosure provides an overview of test components and systems that on their own or in combination represent test options that could be used to direct care of UTIs in clinical, practitioner's offices, for home use, or in rural healthcare settings. In a particular embodiment, the disclosure provides for a biochemistry-powered dipstick test (U-DETECT) to support antibiotic therapy selection for complicated urinary tract infections (cUTIs). While there are several existing and emerging technologies that aim to offer antibiotic susceptibility results in hours, U-DETECT is a diagnostic test that is capable of yielding susceptibility results in minutes, by harnessing a biochemical system known as DETECT (Dual-Enzyme Trigger-Enabled Cascade Technology). DETECT is a two-tiered system designed to identify the presence of low abundant enzymes produced by bacteria which enable them to circumvent the therapeutic action of an antibiotic. Tier-1, the targeting tier of DETECT is comprised of a small molecule probe (sometime referred to as a targeting-probe) that mimics the antibiotic scaffold applied as the first line therapy for UTI. If a UTI is caused by a drug resistant bacterium (uropathogen), the targeting-probe will be hydrolyzed and will liberate a triggering unit, that will trigger the signaling tier (Tier-2) of DETECT, generating a chromogenic signal output. The chromogenic signal generated by DETECT is analogous to the growth of bacteria observed using standard antibiotic susceptibility tests, whereby color signal generation would indicate the presence of a resistant infection to a given antibiotic, and the absence of color would indicate susceptibility. The disclosure provides for a rapid diagnostic dipstick test that utilizes DETECT for detecting antibiotic resistant bacteria, and methods of use and methods of making thereof.

In a particular embodiment, the disclosure provides a solid substrate comprising reagents or components for measuring the presence of a β-lactamase produced by a Gram-negative, Gram-positive or pathogenic beta-lactam resistant bacteria, comprising a first pad on the solid substrate which comprises a targeting small molecule probe that is specificity acted on by a β-lactamase; a caged enzyme amplifier; and a chromophore-releasing small molecule indicator that is activated by an uncaged enzyme amplifier. In another embodiment, the solid substrate comprises a second pad on the solid substrate which comprises the caged enzyme amplifier; and the chromophore-releasing small molecule indicator that is activated by the uncaged enzyme amplifier, wherein the second pad does not comprise the targeting small molecule probe, optionally, a third or more pads on the solid substrate which comprises reagents or components. In still another or further embodiment, the components of the first pad and/or second pad are dried. In still another or further embodiment, the first pad, the second pad, and the optional third or more pads comprise an adsorbent or plastic material. In a further embodiment, the adsorbent material is selected from paper, an adsorbent polymer, silica gel, glass fiber, zeolite or a fabric. In still a further embodiment, the paper is selected from cellulose, nitrocellulose, Teslin, or filter paper. In still another embodiment, the plastic material is selected from the group consisting of high-density polyethylene (HDPE), low-density polyethylene (LDPE), polypropylene (PP), polyvinyl chloride (PVC), polycarbonate (PC), polysulfone (PSF), polystyrene (PS), Teflon (FEP), or Teflon (PFA). In any of the foregoing embodiments, the solid substrate comprises paper or plastic. Yet in another embodiment of any of the foregoing embodiments, the solid substrate is a rectangular strip that is less than 10 millimeters wide. In still another embodiment of any of the foregoing, the targeting small molecule probe comprises a β-lactam group. In still another embodiment of any of the foregoing, when the targeting small molecule probe is acted upon by a β-lactamase the targeting small molecule probe releases a thiophenol group that interacts with the caged enzyme amplifier to uncage and activate the enzyme amplifier. In still further embodiments of any of the foregoing, the caged enzyme amplifier is a caged cysteine protease. In a further embodiment, the cysteine protease is selected from papain, bromelain, cathepsin K, calpain, caspase-1, separase, adenain, pyroglutamyl-peptidase I, sortase A, hepatitis C virus peptidase 2, sindbis virus-type nsP2 peptidase, dipeptidyl-peptidase VI, deSI-1 peptidase, TEV protease, amidophosphoribosyltransferase precursor, gamma-glutamyl hydrolase, hedgehog protein, and dmpA aminopeptidase. In still another embodiment of any of the foregoing, the caged enzyme amplifier is a caged papain enzyme. In a further embodiment, the caged papain has the structure of papain-S—S—CH₃. In another embodiment of any of the foregoing, the chromophore-releasing small molecule indicator is a N-benzoyl-DL-arginine-4-nitroanilide (BAPA) or a derivative thereof. In a further embodiment, the derivative of BAPA comprises a different chromophore group for a p-nitroaniline group. In still a further embodiment, the derivative of BAPA comprises the dipeptide —X₁X₂— or the tripeptide —X₁X₂X₃— in place of the arginine group of BAPA, wherein X₁, X₂ and X₃ are each independently selected from any amino acid. In yet a further embodiment, at least one of X₁ to X₃ is a hydrophobic amino acid. In a further embodiment, one of X₁ to X₃ is a phenylalanine. In still another embodiment of any of the foregoing embodiments, a chromophore is released from the chromophore-releasing small molecule indicator when acted on by the enzyme amplifier. In a further embodiment, the chromophore absorbs light in a wavelength from 350 nm to 900 nm. In still a further embodiment, the chromophore is p-nitroaniline and absorbs light in a wavelength of about 405 nm. In another or further embodiment, the chromophore absorbs light in a wavelength from 520 nm to 600 nm. In a further embodiment, the chromophore is resorufin. In still another embodiment of any of the foregoing, the solid substrate comprises a third pad affixed onto the solid substrate which comprises the enzyme amplifier; and the chromophore-releasing small molecule indicator that is activated by the enzyme amplifier. In another or further embodiment of any of the foregoing embodiments, the solid substrate comprises a fourth pad affixed onto the solid substrate which comprises reagents or components including the chromophore-releasing small molecule indicator that is activated by the enzyme amplifier.

The disclosure also provides a solid substrate comprising reagents or components for measuring the presence of a β-lactamase produced by a Gram-negative, Gram-positive or pathogenic beta-lactam resistant bacteria, comprising a solid substrate having (a) a first region or layer comprising one or two components selected from the group consisting of (i) a targeting small molecule probe that is acted upon by a beta-lactamase; (ii) a caged enzyme amplifier; and (iii) a chromophore-releasing small molecule indicator; and (b) a second region or layer comprising (i), (ii) and/or (iii), not present in the first region. In one embodiment, the first region and second region are separated linearly from one another along a path followed by a wicked-liquid (e.g., a lateral flow assay system). In another embodiment, the first region and second region are layered upon one another. In still another or further embodiment, the solid substrate comprises a third region having the caged enzyme amplifier; and the chromophore-releasing small molecule indicator but does not comprise the targeting small molecule probe; optionally, a fourth or more region on the solid substrate which comprises reagents or components. In still another or further embodiment, components of the first, second or third regions are dried. In still another or further embodiment, the components of the first, second or third region are absorbed or adsorbed to the solid substrate or a material attached thereto. In a further embodiment, the solid support or material is selected from paper, an adsorbent polymer, a plastic a silica gel, a glass fiber, a zeolite or a fabric. In still a further embodiment, the paper is selected from cellulose, nitrocellulose, Teslin, or filter paper. In still a further embodiment, the plastic material is selected from the group consisting of high-density polyethylene (HDPE), low-density polyethylene (LDPE), polypropylene (PP), polyvinyl chloride (PVC), polycarbonate (PC), polysulfone (PSF), polystyrene (PS), Teflon (FEP), or Teflon (PFA). Yet in another embodiment of any of the foregoing embodiments, the solid substrate is a rectangular strip that is less than 10 millimeters wide. In still another embodiment of any of the foregoing, the targeting small molecule probe comprises a β-lactam group. In still another embodiment of any of the foregoing, when the targeting small molecule probe is acted upon by a β-lactamase the targeting small molecule probe releases a thiophenol group that interacts with the caged enzyme amplifier to uncage and activate the enzyme amplifier. In still further embodiments of any of the foregoing, the caged enzyme amplifier is a caged cysteine protease. In a further embodiment, the cysteine protease is selected from papain, bromelain, cathepsin K, calpain, caspase-1, separase, adenain, pyroglutamyl-peptidase I, sortase A, hepatitis C virus peptidase 2, sindbis virus-type nsP2 peptidase, dipeptidyl-peptidase VI, deSI-1 peptidase, TEV protease, amidophosphoribosyltransferase precursor, gamma-glutamyl hydrolase, hedgehog protein, and dmpA aminopeptidase. In still another embodiment of any of the foregoing, the caged enzyme amplifier is a caged papain enzyme. In a further embodiment, the caged papain has the structure of papain-S—S—CH₃. In another embodiment of any of the foregoing, the chromophore-releasing small molecule indicator is a N-benzoyl-DL-arginine-4-nitroanilide (BAPA) or a derivative thereof. In a further embodiment, the derivative of BAPA comprises a different chromophore group for a p-nitroaniline group. In still a further embodiment, the derivative of BAPA comprises the dipeptide —X₁X₂— or the tripeptide —X₁X₂X₃— in place of the arginine group of BAPA, wherein X₁, X₂ and X₃ are each independently selected from any amino acid. In yet a further embodiment, at least one of X₁ to X₃ is a hydrophobic amino acid. In a further embodiment, one of X₁ to X₃ is a phenylalanine. In still another embodiment of any of the foregoing embodiments, a chromophore is released from the chromophore-releasing small molecule indicator when acted on by the enzyme amplifier. In a further embodiment, the chromophore absorbs light in a wavelength from 350 nm to 900 nm. In still a further embodiment, the chromophore is p-nitroaniline and absorbs light in a wavelength of about 405 nm. In another or further embodiment, the chromophore absorbs light in a wavelength from 520 nm to 600 nm. In a further embodiment, the chromophore is resorufin.

The disclosure also provides a method to measure the presence of a β-lactamase produced by a pathogen in a sample, comprising: contacting the solid substrate as set forth in any of the embodiments above and described hereinbelow with the sample, and measuring light absorbance at a wavelength from 400 nm to 600 nm, wherein a measured change in light absorbance of the solid substrate is indicative that there is a β-lactamase produced by a pathogen in the sample. In one embodiment, the β-lactamase is selected from TEM-1, SHV-1, CTX-M-14, CTX-M-15, CMY-2, and KPC-2. In another or further embodiment, the pathogen is E. coli, K. pneumoniae, Pseudomonas aeruginosa and/or P. mirabilis. In another embodiment, the sample is from a subject. In still another embodiment, the sample is a urine or blood sample. In a further embodiment, the sample is a urine sample from a subject suspected of having a urine infection.

In other embodiments of the disclosure the solid substrate or method as described herein, comprises a targeting small molecule probe having the general structure of Formula I:

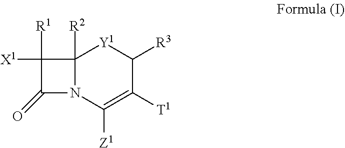

or a salt, stereoisomer, tautomer, polymorph, or solvate thereof, wherein: T¹ is a benzenethiol containing group; Z¹ is a carboxylate, a carbonyl, or an ester; X¹ is

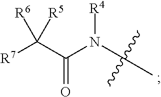

### Y¹ is

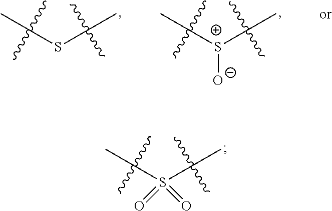

R¹-R⁶ are each independently selected from H, D, hydroxyl, nitrile, halo, amine, nitro, amide, thiol, aldehyde, carboxylic acid, alkoxy, optionally substituted (C₁-C₄) ester, optionally substituted (C₁-C₄) ketone, optionally substituted (C₁-C₆)alkyl, optionally substituted (C₁-C₆)alkenyl, optionally substituted (C₁-C₆)alkynyl, optionally substituted (C₅-C₇) cycloalkyl, optionally substituted aryl, optionally substituted benzyl, and optionally substituted heterocycle; R⁷ is an optionally substituted (C₅-C₇) cycloalkyl, optionally substituted aryl, optionally substituted benzyl, or optionally substituted heterocycle. In a further embodiment, T¹ is a benzenethiol group selected from the group consisting of:

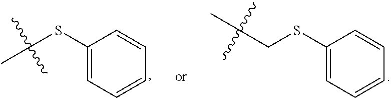 [SMILES: *Sc1ccccc1 ; *CSc1ccccc1]

In still another embodiment, R⁷ is selected from the group consisting of:

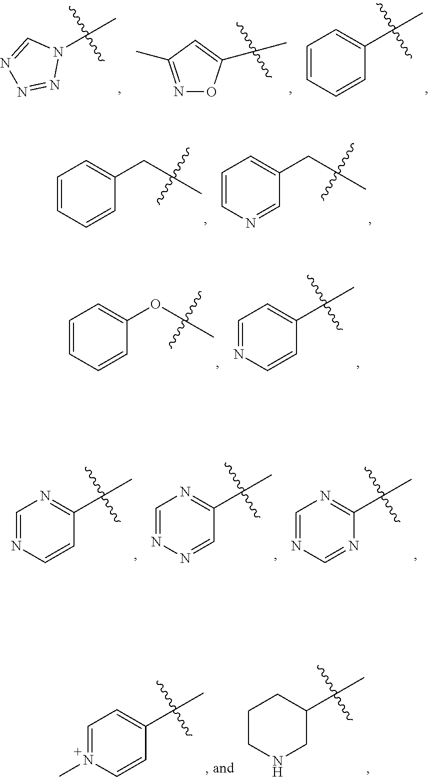

In yet another embodiment, the compound has a structure of Formula I(a):

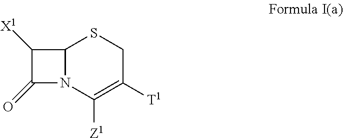

or a salt, stereoisomer, tautomer, polymorph, or solvate thereof, wherein T is a benzenethiol containing group; Z¹ is a carboxylate, a carbonyl, or an ester; X¹ is

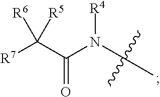

### Y¹ is

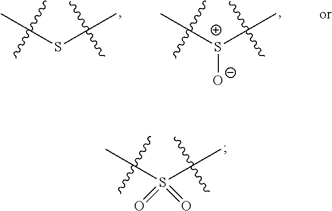

R¹-R⁶ are each independently selected from H, D, hydroxyl, nitrile, halo, amine, nitro, amide, thiol, aldehyde, carboxylic acid, alkoxy, optionally substituted (C₁-C₄) ester, optionally substituted (C₁-C₄) ketone, optionally substituted (C₁-C₆)alkyl, optionally substituted (C₁-C₆)alkenyl, optionally substituted (C₁-C₆)alkynyl, optionally substituted (C₅-C₇) cycloalkyl, optionally substituted aryl, optionally substituted benzyl, and optionally substituted heterocycle; R⁷ is an optionally substituted (C₅-C₇) cycloalkyl, optionally substituted aryl, optionally substituted benzyl, or optionally substituted heterocycle. In a further embodiment, T¹ is a benzenethiol group selected from the group consisting of:

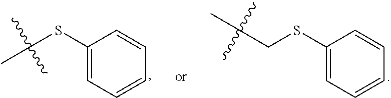 [SMILES: *Sc1ccccc1 ; *CSc1ccccc1]

In still a further embodiment, R⁷ is selected from the group consisting of:

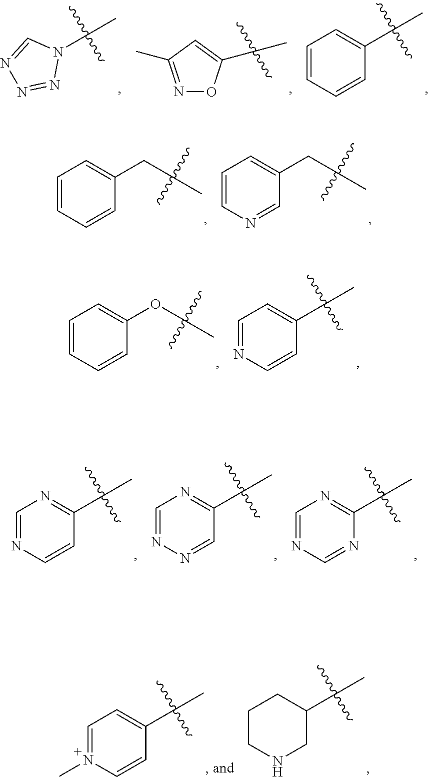

In yet another embodiment, the compound has the structure of Formula I(b):

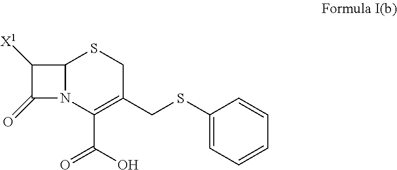

### X¹ is

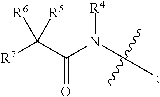

### Y¹ is

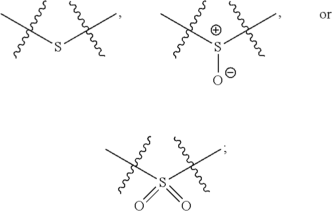

R¹-R⁶ are each independently selected from H, D, hydroxyl, nitrile, halo, amine, nitro, amide, thiol, aldehyde, carboxylic acid, alkoxy, optionally substituted (C₁-C₄) ester, optionally substituted (C₁-C₄) ketone, optionally substituted (C₁-C₆)alkyl, optionally substituted (C₁-C₆)alkenyl, optionally substituted (C₁-C₆)alkynyl, optionally substituted (C₅-C₇) cycloalkyl, optionally substituted aryl, optionally substituted benzyl, and optionally substituted heterocycle; R⁷ is an optionally substituted (C₅-C₇) cycloalkyl, optionally substituted aryl, optionally substituted benzyl, or optionally substituted heterocycle. In a further embodiment, T¹ is a benzenethiol group selected from the group consisting of:

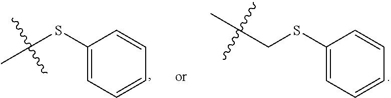 [SMILES: *Sc1ccccc1 ; *CSc1ccccc1]

In a further embodiment, R⁷ is selected from the group consisting of:

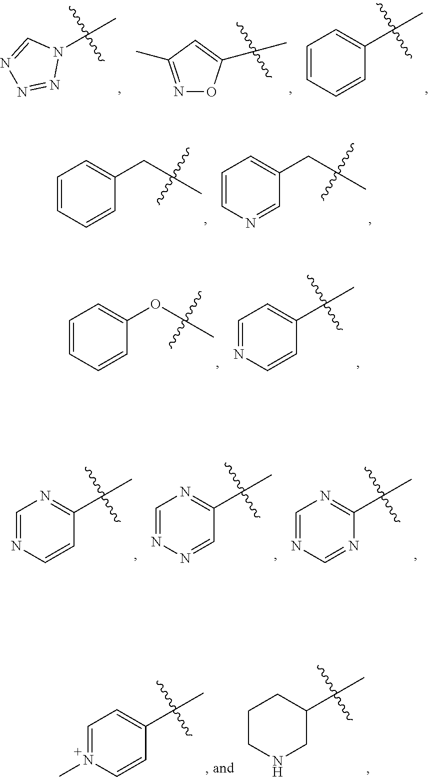

In still another or further embodiment of any of the foregoing, the targeting small molecule probe comprises a compound selected from the group consisting of

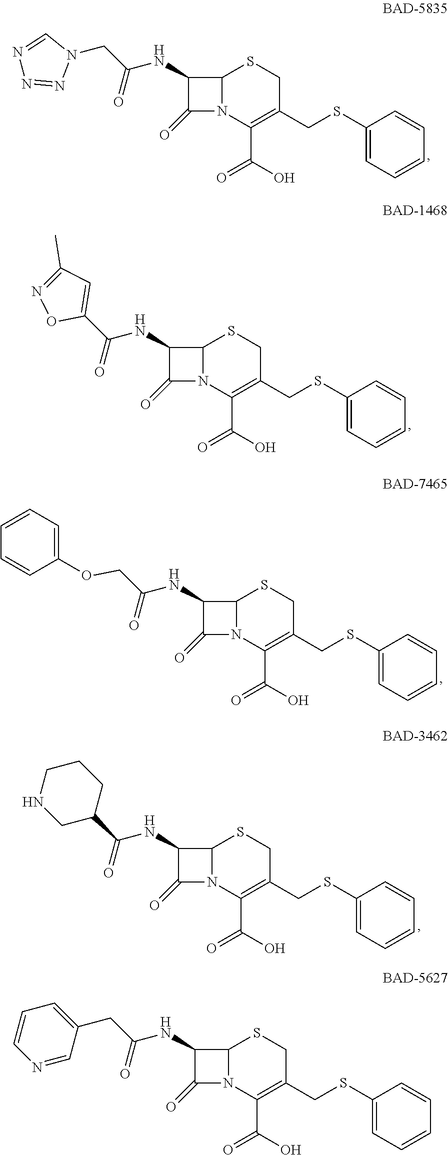

or a salt, stereoisomer, tautomer, polymorph, or solvate thereof.

The invention encompasses all combinations of the particular embodiments recited herein, as if each combination had been laboriously recited.

The details of one or more embodiments of the disclosure are set forth in the accompanying drawings and the description below. Other features, objects, and advantages will be apparent from the description and drawings, and from the claims.

### DESCRIPTION OF DRAWINGS

FIG. 1A-B provides (A) an overview of an embodiment of a DETECT assay that can be applied to reveal CTX-M β-lactamase activity directly in clinical urine samples. A representation of the experimental workflow applied to analyze a urine sample by DETECT. A small volume of urine is transferred into a well containing DETECT reagents (D; steps 1 and 2). The absorbance at 405 nm (A_{405 nm}) is recorded with a spectrophotometer at 0 min. If the target resistance marker is present (E1; a CTX-M ESBL enzyme) the targeting probe is hydrolyzed and the thiophenol trigger eliminates from the probe, subsequently activating the amplification and colorimetric signal output tier of DETECT (step 3). After 30 min of room temperature incubation an A_{405 nm} reading is again recorded, and the DETECT score is calculated (step 4; A_{405 nm} T30-T0). A DETECT score exceeding an experimentally determined threshold value indicates the sample contains the target CTX-M β-lactamase, and hence, an expanded-spectrum cephalosporin-resistant GNB is present in the urine sample (step 5). A DETECT score that is lower than the threshold value indicates the sample does not contain the target resistance marker. BAPA: Nα-Benzoyl-L-arginine 4-nitroanilide hydrochloride. (B) chemical structures of targeting probe options that can be used in the DETECT assay or used as dry reagent components; shows use of reagents BAD-5385 (G2B) and BAD-5627 with sensitivity towards β-lactamases as a measure of absorbance.

FIG. 2 shows embodiments of components for the U-DETECT test strip and the possible test results. As shown, are listing of components that could be embedded/impregnated into the “sample” pad: sodium phosphate buffer system, Bis Tris buffer system, modified enzyme amplifier (such as a cysteine protease), indicator small molecule (e.g., N-benzoyl-L-arginine-nitroaniline, or derivatives thereof.) and a BAD probe (defined as a small molecule probe molecule comprised of a thiophenol moiety that is selectively liberated by a complementary enzyme biomarker). All components can be prepared to 1-5-X concentration relative to the standard concentration applied for the liquid phase assay version of the test. Description of the potential components that could be embedded/impregnated into the “control” pad, which are the same as the components described in the “sample” pad with exclusion of BAD probe. The pads described are a solid support denoted as “paper” wherein “paper” can include but not limited to, nitrocellulose, cellulose, glass fiber, Teslin, filter paper etc.

FIG. 3A-H presents embodiments of potential test strip formats. (A) Each test requires a “blank” and “sample” pad to yield a result outcome (+/−). When an analyzer is applied to measure the reflectance or fluorescence of the surface then a quantitative value could be gained. The test operates whereby a discernable difference in the visual color, fluorescence or reflectance is greater in the “sample” pad relative to the “control” pad. A-D represent the combination of positive and negative control pads that could be included in a complete test strip, where a “positive” pad would include a standard thiol trigger component and a “negative” pad that excludes modified enzyme and a small molecule biomarker-targeting probe. (E-F) To develop tests that can differentiate families of a target biomarker (e.g., β-lactamases) inhibitor compounds can be incorporated into a test strip as described in E. Combinations of “positive” and “negative” control pads that could be included are comprised across F—H.

FIG. 4 demonstrates how coatings of the “sample” and “control” pads can be performed by preparing a “sample” solution comprised of the components described in the “sample” pad of FIG. 2 and a “control” solution comprised of the components also described in the “sample” pad of FIG. 2 and coating a “paper” pad surface using an auto-dispensing, fabric or rubber stamp, dropper, inkjet printer or derivatives thereof, or marker. Coating of the pads in parallel or succession would then be followed by an applied drying method that could include but are not limited to forced air, lyophilization or thermal drying.

FIG. 5 indicates embodiments where components that require separation of aqueous compatible and incompatible components two (2), three (3), or four (4) individual solutions couple be prepared, coated onto a “paper” pad. The order could proceed as “sample” organic solution coating onto a sample paper pad, drying of sample pad, “blank” organic solution coating on blank paper pad, drying of control pad, coating of sample and blank aqueous solutions (could also be prepared as one solution) onto sample and control pads simultaneously and then a final drying step. The figure also depicts a more generic preparation option which can be utilized to prepare for coating or strips or coating of a well.

FIG. 6A-B provides an embodiment of a flow chart of the diagram in FIG. 5 on how coatings of the “sample”, “control”, or other types of pads can be sequentially (A) applied using drying steps after each coating application set or simultaneously (B) applied and dried thereafter.

FIG. 7 provides an embodiment of a simplified workflow of applying the liquid phase reagents (R1 & R2) onto the reagent pad and its subsequent drying, cutting, and adhering steps

FIG. 8A-B provides an overview of Study 1 and Study 2 and the corresponding solution each strip received. Study 1 materials: Papain, BAPA, BAD-5835 and cysteine (100 μM). Study 2 materials: Papain, BAPA, BAD-5835 and CTX-M-15 (1 nM or 4 nM). Left pain: representation of each study and the test strips that represent the control (buffer) and sample (cysteine or CTX-M-15). Right pane: Percent reflectance of the sample strip is subtracted from the control strip for study 1 (A) and study 2 (B) for each time point.

FIG. 9A-D presents the results: (A) % Rscore (% reflectance control-% reflectance sample) over time for formulations 1-4 of Table 1. (B) The DETECT % Rscore (% Rscore_{20 min}−% Rscore_{2 min}) for formulation 1-4 of Table 1. (C) % Rscore (% reflectance control-% reflectance sample) for formulation 2 & 5-9 of Table 1 over time. (D) The DETECT % Rscore (% Rscore_{20 min}−% Rscore_{2 min}) for formulation 2 & 5-9 of Table 1.

FIG. 10A-D presents reflectance data for 1 nM (A) and (B); and 4 nM (C) and (D); of CTX-M-15. Where the % Rscore is the % reflectance control-% reflectance sample for each time point. And the DETECT % Rscore is the % Rscore_{20 min}−% Rscore_{2 min} for formulation 7-9 of Table 1.

FIG. 11A-D shows the ability of the tri- and dipeptide substrates to provide significant absorbance change (DETECT score) and maintain high signal-to-noise (S/N) at 5 minutes. (A) Kinetic parameters for each papain substrate obtained at ambient temperature by addition of cysteine to activate papain and monitoring the increase of absorbance at 405 nm. (B) Absorbance change (DETECT score) observed from t=0 to t=5 m upon papain activation by cysteine, and (C) S/N, where signal is absorbance generated in the presence of cysteine and noise is amount of absorbance of signal generated in the absence of the cysteine papain activator. (D) Shows the chemical structures of BAPA derivatives including the di and tri-peptide substrates Bz-FR and Bz-QFR.

FIG. 12A-C presents the DETECT scores calculated for (A) DETECT 1.0 and (B) DETECT-BzFR for each isolate (10⁶ CFU/mL). The DETECT score is determined by calculating the difference between sample and control wells after 5 minutes of assay incubation (AbsΔ_{5 min}−AbsΔ_{0 min}). (C) The % increase in signal relative to noise was calculated using the raw absorbance values after 5 minutes of assay incubation for each isolate. The noise used for this calculation is the amount of absorbance that is generated for the negative (SF505) isolate, where % increase=(Abs_{signal}−Abs_{negative})/Abs_{negative}*100.

FIG. 13 provides an example of a chromogenic probe for sulfite, where a drastic color is obtained when the protected resorufin reacts with sulfite.

FIG. 14A-B provides an overview of the transformation that the BAPA derivatives will undergo to provide a longer wavelength signaling molecule for papain. Conversion of (A) Bz-FR-pNA and of the (B) Bz-XX-pNa candidate to its respective resorufin derivative. XX=optimal amino acids (R1 and R2).

FIG. 15A-B provides (A) an overview of the synthesis plan to obtain azo-based chromophore derivatives. Where X═H, NO₂, Cl, or Br and R₁═H, OCH₃, SO₃⁻. The NH₂ group is highlighted to denote the reactive group for C-terminal modification. (B) Modification of the identified peptide backbone Bz-XX—COOH to yield the derivative BAPA-Azo papain substrate.

FIG. 16A-B presents (A) proposed indoaniline chromophore scaffolds that will be synthesized and characterized. (B) Modification of the identified peptide backbone Bz-XX—COOH to yield the derivative BAPA-Indo papain substrate.

### DETAILED DESCRIPTION

As used herein and in the appended claims, the singular forms “a,” “an,” and “the” include plural referents unless the context clearly dictates otherwise. Thus, for example, reference to “a β-lactamase substrate” includes a plurality of such substrates and reference to “the f-lactamase” includes reference to one or more -lactamases and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated herein by reference in full for the purpose of describing and disclosing the methodologies, which might be used in connection with the description herein. Moreover, for terms expressly defined in this disclosure, the definition of the term as expressly provided in this disclosure will control in all respects, even if the term has been given a different meaning in a publication, dictionary, treatise, and the like.

Extended-spectrum β-lactamase (ESBL)-producing Gram-negative bacteria (GNB) express enzymes that hydrolyze and inactivate most β-lactam antibiotics, including penicillins, cephalosporins, expanded-spectrum cephalosporins (including 3^{rd} and 4^{th}-generation agents), and monobactams. ESBL-producing Enterobacteriaceae were designated a “serious threat” by the Centers for Disease Control and Prevention (CDC) in their Antibiotic Resistance Threats report in 2013 and 2019, and a “critical priority” by the World Health Organization in their Global Priority List of Antibiotic-Resistant Bacteria in 2017. In 2017 there were an estimated 197,400 ESBL-producing Enterobacteriaceae infections in hospitalized patients in the United States, resulting in 9,100 deaths and $1.2 B in attributable healthcare costs. ESBL infections represent a major public health concern—infections occur in both healthcare and community settings, and their prevalence is increasing in the US and globally.

Due to their low toxicity and efficacy against a range of clinically relevant bacteria, β-lactams are one of the most important classes of antibiotics, comprising ˜50% of the total antibiotic prescriptions worldwide. This antibiotic class is comprised of generations of variants that differ by only the chemical pendants decorated across two centers of the β-lactam pharmacophore (C3 and C7). The predominant mechanism of resistance which has evolved within bacteria to circumvent the therapeutic action of β-lactams, is through the production of β-lactamase enzymes that work by hydrolyzing of the β-lactam pharmacophore. Interestingly, there are thousands of β-lactamase variants that are grouped and classified by the phenotypic resistance that results from their expression. For example, first-generation β-lactamases (ex. TEM-1, SHV-1, and OXA-1) are grouped and defined based on their ability to yield resistance to early generation β-lactams. As such, uropathogens that produce first-generation β-lactamases will be resistant to the earliest-generations of β-lactams, such as ampicillin.

The clinical management of cUTI has become increasingly problematic due to a rising prevalence of antibiotic resistant bacteria, known as extended spectrum-β-lactamase (ESBL) producing uropathogens (˜15-20% prevalence across the US), that are resistant to the first-line therapy for cUTI, ceftriaxone (a third generation β-lactam). Moreover, ESBL-uropathogens are often multidrug resistant (MDR), further reducing treatment options for these infections. In a retrospective study which was in partnership with Kaiser Permanente, the impact of delayed time to diagnosis of ESBL-associated cUTIs was elucidated. In this study, patient cohorts were divided into ESBL-associated (case), versus non-ESBL-associated cUTIs (control) groups to investigate the impact of ESBL producing uropathogens (E. coli and Klebsiella) on the outcomes of patients hospitalized for a cUTI. It was observed that 63% of ESBL-associated cUTIs were treated with an “inappropriate” first-line therapy, resulting in hospital stays that were 1.2-times longer than patients with non-ESBL cUTI, and had 33% higher mortality rates (65/530 case vs 279/3,577 control). Given that there is no precedence for increased virulence of ESBL-producing organisms, the single factor attributed to poorer outcomes of the case versus the control group is delayed time to diagnosis. Therefore, a diagnostic test that can immediately identify an ESBL-associated cUTI to establish appropriate therapy would yield improved outcomes for patients experiencing a cUTI. Moreover, 16% of non-ESBL-associated cUTIs are unnecessarily treated with a broad-spectrum carbapenem, which are a class of last resort antibiotics usually reserved for serious infections that have few other therapeutic options available.

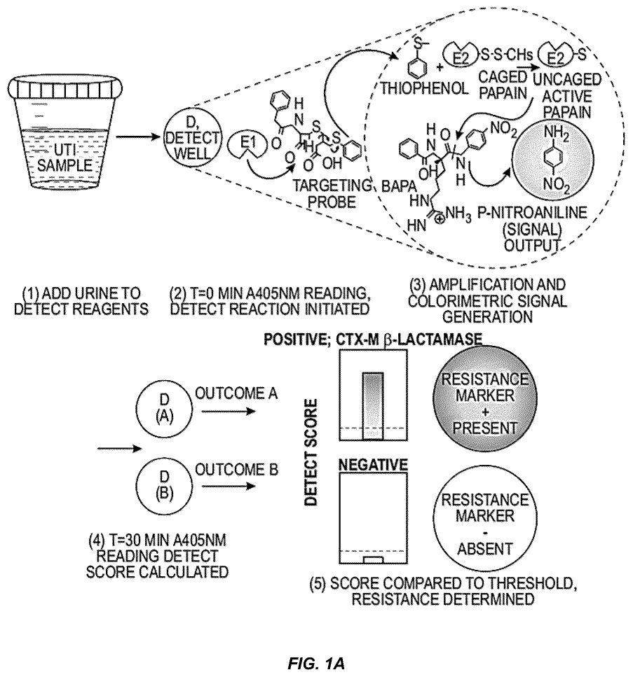

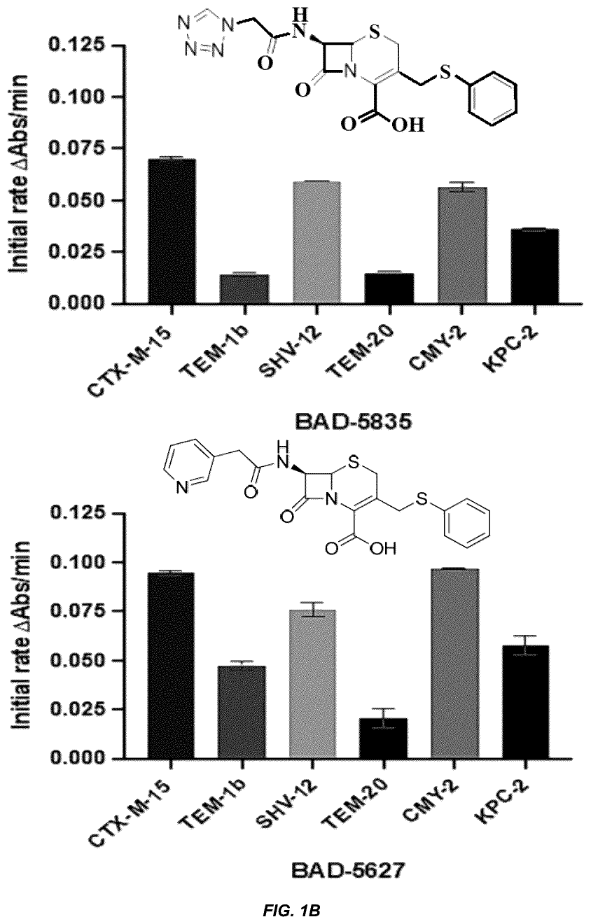

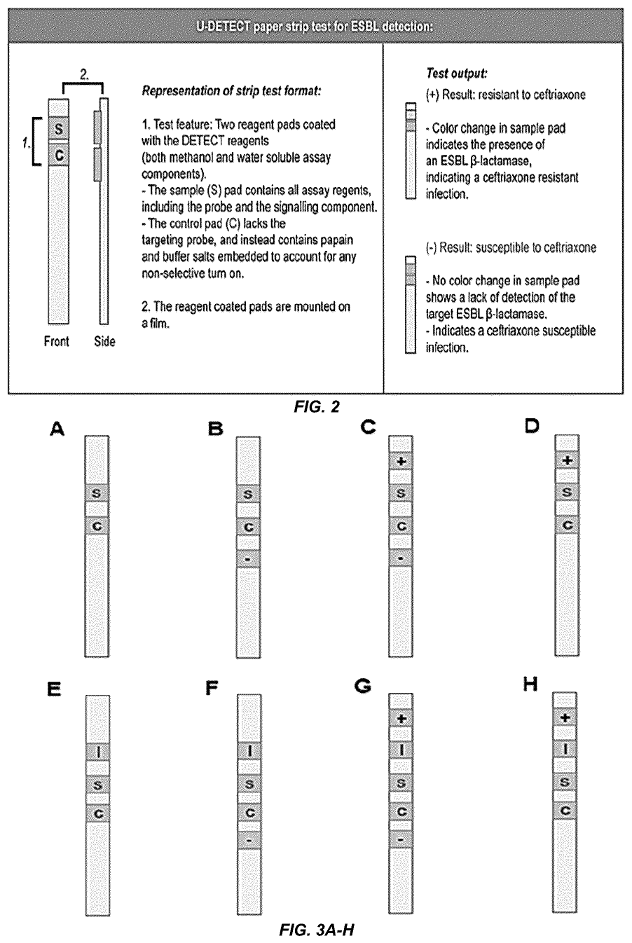

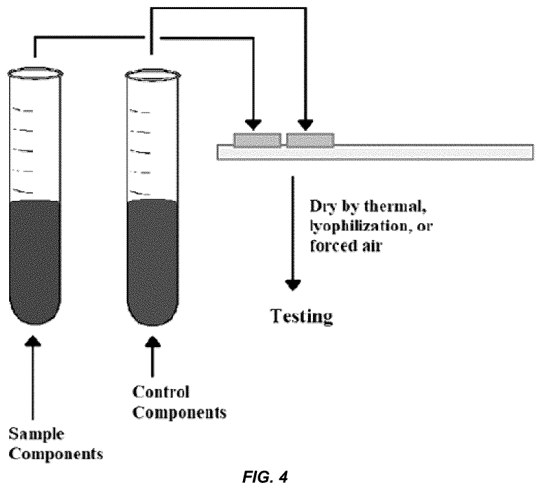

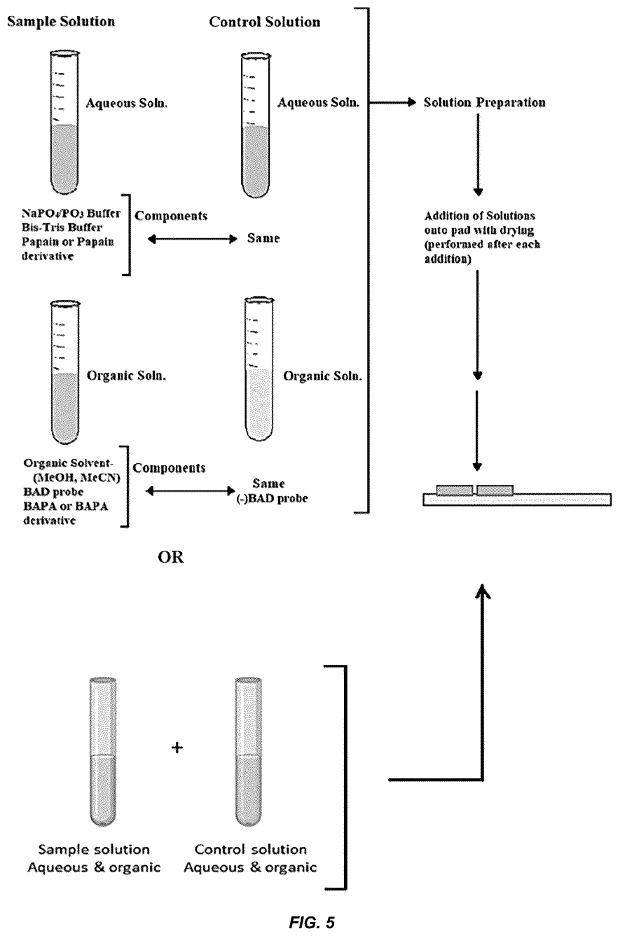

The disclosure provides for the use of a DETECT assay in a reagent test strip format (e.g., a dry reagent test strip format). As shown in FIG. 1, DETECT is a solution-based assay that utilizes amplification technology with a dual enzyme cascade. In particular, DETECT uses a targeting probe to target a resistance marker (E1; a CTX-M ESBL enzyme), and if the target resistance marker is present the targeting probe is hydrolyzed and the thiophenol trigger eliminates from the probe, subsequently activating the amplification and colorimetric signal output tier of DETECT. DETECT has been used to identify ESBL-producing uropathogens as a proxy for ceftriaxone and cefotaxime resistance/susceptibility, directly from unprocessed clinical urine samples. In a double-blinded study, 472 clinical urine samples were analyzed in a solution phase assay test and demonstrated the diagnostic performance of DETECT to be 90.9% sensitive and 97.6% specific, with a negative predictive value (NPV) of 99.7 and a positive predictive value (PPV) of 45.4% (AUC 0.937; 95% confidence interval, 0.822-1.000). These results were relative to the clinical reference standard of bacterial culture and antibiotic susceptibility testing (C&S).

The introduction of dry reagent test strips in the 1950's revolutionized the way clinical testing was conducted. Tests that were routinely conducted in clinical chemistry laboratories shifted to other healthcare departments such as physician's offices, outpatient clinics, and even by the patient at home. This evolution was attributed to the success of the first semi-quantitative urinary glucose test strip, which set the precedence for the development of test strips for other analytes (protein, ketones, bilirubin, nitrite, esterase, and pH) found in urine, that have now become the standard practice of multi-panel tests for urinalysis. The low cost and simplicity of dry reagent test strips were fundamental reasons for their wide adoption. Additionally, these tests can be performed directly on patient urine samples by unskilled users, and with results being produced in under 5 minutes.

The fabrication of dry reagent test strips involves controlled application of reagents (i.e., indicator dye, enzymes, small molecule probes, polymers etc.) onto a solid matrix, such as cellulose, and dried with heating. After the drying process, the solid matrix is bonded with adhesive onto a plastic support, which allows the test to be carried by the user and dipped into the sample of interest (alternatively a sample can be added to a reagent pad). Results are subsequently read on a reflectance spectrophotometer instrument. Translation of several enzymatic assays (solution phase) to dry reagent test strips has offered several advantages over the assay form, such as convenience, reduced minimum storage requirements (i.e., cold chain), and in some instances, improved stability of reagents. Additionally, dry reagent test strips eliminate multi-step procedures that normally require a skilled user, enabling results with minimal processing steps. Economically, reagent test strips are cost-effective and minimally requires a hand-held reflectance instrument (for quantitative measurements). It is well understood that certain assay reagents can be too unstable to undergo the fabrication process and would require optimization for reagent test strip development.

As such, the dry reagent test strips disclosed herein (e.g., U-DETECT) not only offer improved outcomes for patients and subjects, but will also support sparing of broad-spectrum antibiotics that drive the spread of infections resistant to last-resort antibiotic agents. In a particular embodiment, the dry reagent test strips disclosed herein comprise a thiophenol-releasing small molecule probe, an enzyme amplifier (such as a cysteine protease) and chromophore-releasing small molecule indicator that is activated by the enzyme amplifier. Specific examples of thiophenol-releasing targeting small molecule probes are described in PCT/US2020/048060, the disclosure of which is incorporated by reference in its entirety. Further examples of targeting small molecule probe include those described in the Examples section, including. but not limited to: (7R)-7-(2-(1H-tetrazol-1-yl)acetamido)-8-oxo-3-((phenylthio)methyl)-5-thia-1-azabicyclo[4.2.0]oct-2-ene-2-carboxylic acid (BAD-5835), (7R)-7-(3-methylisoxazole-5-carboxamido)-8-oxo-3-((phenylthio)methyl)-5-thia-1-azabicyclo[4.2.0]oct-2-ene-2-carboxylic acid (BAD-1468), (7R)-8-oxo-3-((phenylthio)methyl)-7-(2-(pyridin-3-yl)acetamido)-5-thia-1-azabicyclo[4.2.0]oct-2-ene-2-carboxylic acid (BAD-5627), and (7R)-8-oxo-3-((phenylthio)methyl)-7-((S)-piperidine-3-carboxamido)-5-thia-1-azabicyclo[4.2.0]oct-2-ene-2-carboxylic acid (BAD-3462), (7R)-8-oxo-7-(2-phenoxyacetamido)-3-((phenylthio)methyl)-5-thia-1-azabicyclo[4.2.0]oct-2-ene-2-carboxylic acid (BAD-7465). For example, a thiophenol-releasing small molecule probe can have the structure of Formula I:

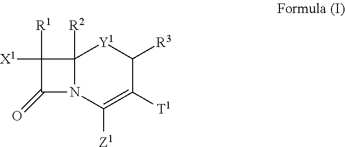

or a salt, stereoisomer (in some instances constitutional isomers), tautomer, polymorph, or solvate thereof, wherein: T is a benzenethiol containing group; Z¹ is a carboxylate, a carbonyl, or an ester; X¹ is

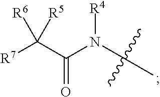

### Y¹ is

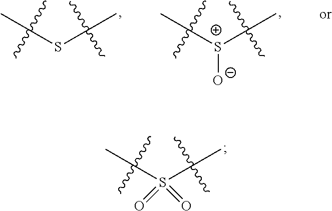

R¹-R⁶ are each independently selected from H, D, hydroxyl, nitrile, halo, amine, nitro, amide, thiol, aldehyde, carboxylic acid, alkoxy, optionally substituted (C₁-C₄) ester, optionally substituted (C₁-C₄) ketone, optionally substituted (C₁-C₆)alkyl, optionally substituted (C₁-C₆)alkenyl, optionally substituted (C₁-C₆)alkynyl, optionally substituted (C₅-C₇) cycloalkyl, optionally substituted aryl, optionally substituted benzyl, and optionally substituted heterocycle; R⁷ is an optionally substituted (C₅-C₇) cycloalkyl, optionally substituted aryl, optionally substituted benzyl, or optionally substituted heterocycle. In another embodiment or a further embodiment of any of the foregoing embodiments, T¹ is a benzenethiol group selected from the group consisting of:

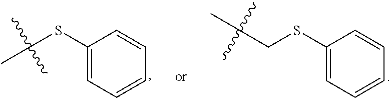 [SMILES: *Sc1ccccc1 ; *CSc1ccccc1]

In another embodiment or a further embodiment of any of the foregoing embodiments, R⁷ is selected from the group consisting of:

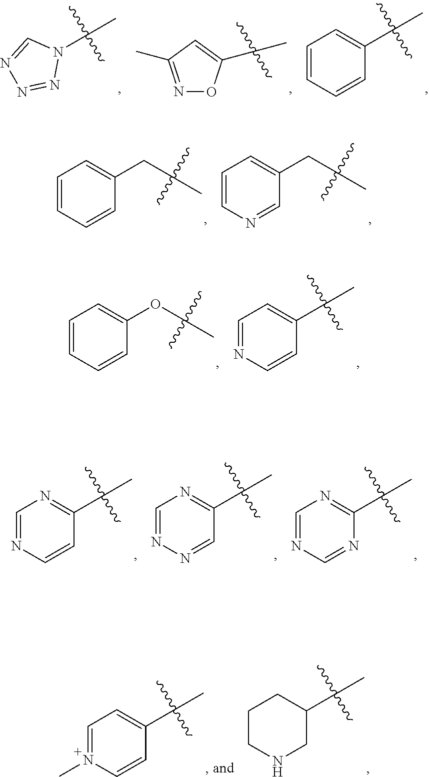

In another embodiment or a further embodiment of any of the foregoing embodiments, the compound has a structure of Formula I(a):

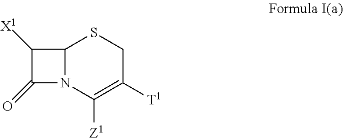

or a salt, stereoisomer, tautomer, polymorph, or solvate thereof, wherein: T¹ is a benzenethiol containing group; Z¹ is a carboxylate, a carbonyl, or an ester; X¹ is

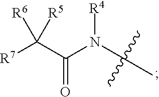

### Y¹ is

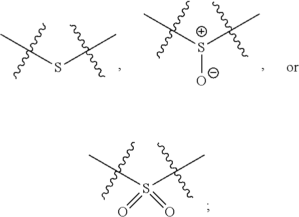

R¹-R⁶ are each independently selected from H, D, hydroxyl, nitrile, halo, amine, nitro, amide, thiol, aldehyde, carboxylic acid, alkoxy, optionally substituted (C₁-C₄) ester, optionally substituted (C₁-C₄) ketone, optionally substituted (C₁-C₆)alkyl, optionally substituted (C₁-C₆)alkenyl, optionally substituted (C₁-C₆)alkynyl, optionally substituted (C₅-C₇) cycloalkyl, optionally substituted aryl, optionally substituted benzyl, and optionally substituted heterocycle; R⁷ is an optionally substituted (C₅-C₇) cycloalkyl, optionally substituted aryl, optionally substituted benzyl, or optionally substituted heterocycle. In another embodiment or a further embodiment of any of the foregoing embodiments, T¹ is a benzenethiol group selected from the group consisting of:

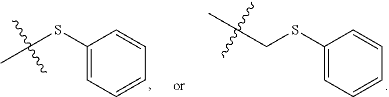 [SMILES: *Sc1ccccc1 ; *CSc1ccccc1]

In another embodiment or a further embodiment of any of the foregoing embodiments, R⁷ is selected from the group consisting of:

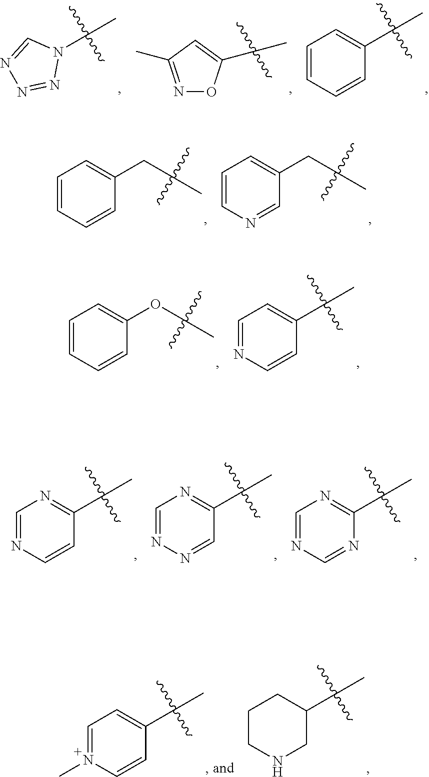

In another embodiment or a further embodiment of any of the foregoing embodiments, the compound has the structure of Formula I(b):

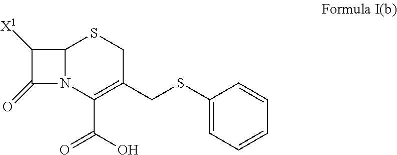

### X¹ is

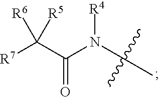

### Y¹ is

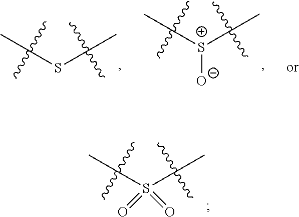

R¹-R⁶ are each independently selected from H, D, hydroxyl, nitrile, halo, amine, nitro, amide, thiol, aldehyde, carboxylic acid, alkoxy, optionally substituted (C₁-C₄) ester, optionally substituted (C₁-C₄) ketone, optionally substituted (C₁-C₆)alkyl, optionally substituted (C₁-C₆)alkenyl, optionally substituted (C₁-C₆)alkynyl, optionally substituted (C₅-C₇) cycloalkyl, optionally substituted aryl, optionally substituted benzyl, and optionally substituted heterocycle; R⁷ is an optionally substituted (C₅-C₇) cycloalkyl, optionally substituted aryl, optionally substituted benzyl, or optionally substituted heterocycle. In another embodiment or a further embodiment of any of the foregoing embodiments, T is a benzenethiol group selected from the group consisting of:

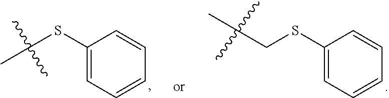 [SMILES: *Sc1ccccc1 ; *CSc1ccccc1]

In another embodiment or a further embodiment of any of the foregoing embodiments, R⁷ is selected from the group consisting of:

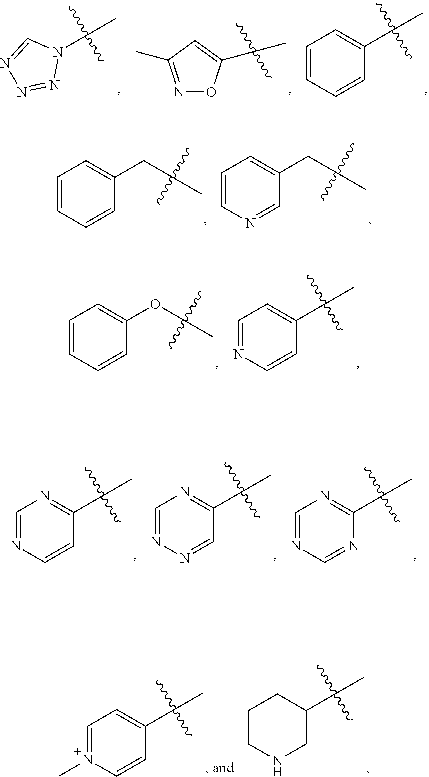

In another embodiment or a further embodiment of any of the foregoing embodiments, the compound is selected from the group consisting of:

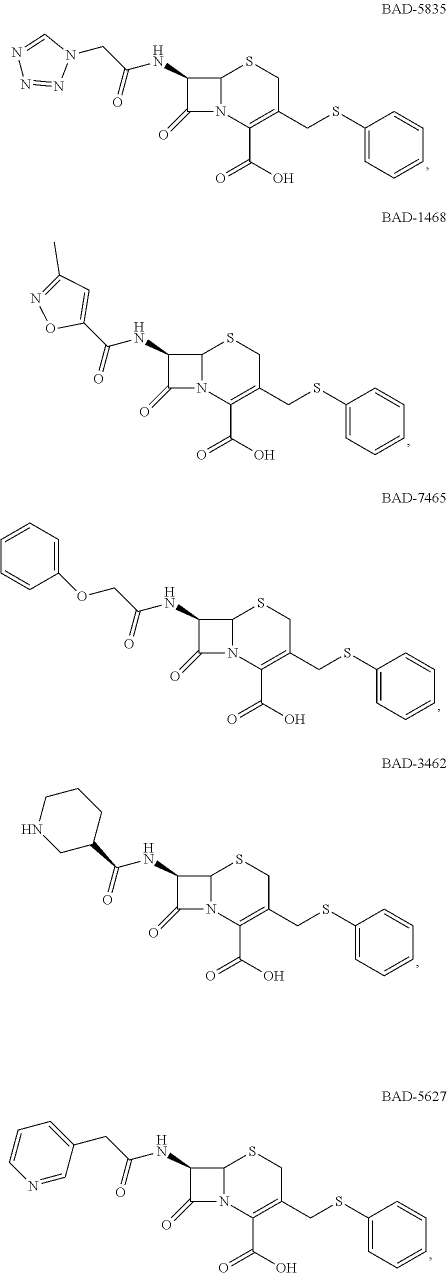

or a salt, stereoisomer, tautomer, polymorph, or solvate thereof.

In another embodiment, the dry reagent test strips disclosed herein comprise a minimum of two (2) test pads that are impregnated with reagent assay components that together can detect the presence of an enzyme biomarker such as a β-lactamase. esterase, cysteine proteases, etc. In a further embodiment, the dry reagent test strips comprise 2, 3, 4, 5, 6, or 7 test pads. or range of test pads that includes or is in between any two of the foregoing. Examples of different types of test pads, can include a positive control, a negative control, a sample test pad, a control test pad lacking a probe, a second, third or fourth control test pad comprising reagent assay components comprising different probes. In a further embodiment, the disclosure also provides that the dry reagent test strips can be read by eye or with a detector device. Examples of a detector device include, but are not limited to, an automated analyzer device, a digital device, a color sensor, or a manual handheld device capable of detecting visible wavelength ranging from 350-950 nm. In yet a further embodiment, the automated analyzer device is a simple urine strip analyzer (e.g., Siemens Clinitek™). In another embodiment, reagent test components are applied to the test pads of the dry reagent test strips. In a further embodiment, the reagent test components are combined or individually contained in the solution phase in reagent containers. In yet a further embodiment, the reagent test components incorporated into an automated dispensing system. In another embodiment, the reagent test components are lyophilized, stamped. chemically or physically entrapped, or coated onto a solid surface (paper, plastic or in a multi-well tray).

The dry reagent test strips and methods of the disclosure have the following features: the assay is rapid and easy to perform and no urine processing steps are required. According, one simply contacts the dry reagent test strip with the sample (e.g., a urine sample) and reads the results in as little as 5 minutes. In view of the following, it is clear that implementation of the method can be carried out at a doctor's office, personnel at a research or clinical bench, or be carried out using semi-automated or fully-automated devices. The dry reagent test strips and methods of the disclosure can be used at the point of care, thereby providing actionable results in a time-frame that positively impacts the identification of a therapeutically effective first antimicrobial agent that can be prescribed to a patient. The dry reagent test strips of the disclosure have a simple colorimetric or other optical output, which should make integration into a device more straightforward and enable flexible format options. The colorimetric output of the compounds and methods of the disclosure can be read by a microplate reader, but could also be read by other spectrophotometric devices or even by a device application (e.g., mobile phone app). Enhancement of the colorimetric signal can also enable accurate detection by eye.

The targeting small molecule probes disclosed herein and in PCT/US2020/048060 (incorporated by reference) are and, as exemplified below, were rapidly hydrolyzed by targeted β-lactamases studied herein. The results demonstrate significant preference of the compounds of the disclosure towards a subclass of ESBLs known as CTX-M-type-lactamases. For example, certain compounds of the disclosure were hydrolyzed by an ESBL to release a trigger unit that activates an enzymes amplifier, initiating an amplification cascade event that generates a colorimetric signal output indicating the presence of an ESBL. The ESBL-detecting compounds can be applied as a diagnostic reagent to detect ESBL-producing pathogens and direct care of patients.

In various aspects, the disclosure provides compounds and methods for detecting antimicrobial resistance via the identification of β-lactamases and variants that are responsible for the enzyme mediated resistance mechanism present in gram-negative and gram-positive bacteria. The targeting small molecule probes provided herein can be formulated into an amplification assay composition that are useful in the disclosed methods. Also provided is the use of the compounds in preparing assay formulations for the amplification method.

The disclosure provides methods to detect the presence of one or more target β-lactamases in a sample by using the reagent test strips disclosure herein. In a particular embodiment, a method disclosed herein has the step of: contacting the test pads of a dry reagent test strip disclosed herein with a sample, and then measuring light absorbance at a wavelength from 350 nm to 950 nm. The sample used in the methods typically is obtained from a subject (including animal subjects), but the sample may also come from other sources, such as a water sample, an environmental sample, food sample, a wastewater sample, etc. Samples obtained from the subject can come from various portions of the body. For example, the sample can be a blood sample, a urine sample, a cerebrospinal fluid sample, a saliva sample, a rectal sample, a urethral sample, or an ocular sample. In regards to the latter three samples these samples can be obtained by swabbing the various regions. In a particular embodiment, the sample is a blood or urine sample. The subject that the sample is obtained from can be from any animal, including but not limited to, humans, primates, cats, dogs, horses, birds, lizards, cows, pigs, rabbits, rats, mice, sheep, goats, etc. In a particular embodiment, the sample is obtained from a human patient that has or is suspected of having a bacterial infection. For example, the human patient may have or be suspected of having a urinary tract infection, sepsis, or other infection.

In regards to targeted β-lactamases, the dry reagent test strips of the disclosure can be used to target every known class of β-lactamases, including subtypes thereof. For example, the compound and methods disclosed herein can be used to delineate and detect the presence of penicillinases, extended-spectrum β-lactamases (ESBLs), inhibitor-resistant β-lactamases, AmpC-type β-lactamases, and carbapenemases. Extended-spectrum β-lactamases or ESBLs, in particular, can be detected by the compounds and methods disclosed herein. For example, the compounds and methods disclosed herein can detect TEM β-lactamases, SHV β-lactamases, CTX-M β-lactamases, OXA β-lactamases, PER β-lactamases, VEB β-lactamases, GES β-lactamases, IBC β-lactamases. As shown in the studies presented herein various compounds disclosed herein can detect CTX-M β-lactamases with high specificity. The compounds and methods disclosed herein can also be used to detect the various subtypes of carbapenemases, including but not limited to, metallo-β-lactamases, KPC β-lactamases, Verona integron-encoded metallo-β-lactamases, oxacillinases, CMY β-lactamases, New Delhi metallo-β-lactamases, Serratia marcescens enzymes, IMIpenem-hydrolysing β-lactamases, NMC β-lactamases and CcrA β-lactamases. For example, the studies presented herein demonstrates that various compounds of the disclosure can detect CMY β-lactamases and KPC β-lactamases with high specificity. In a particular embodiment, compounds disclosed herein can detect CTX-M β-lactamases, CMY β-lactamases and KPC β-lactamases with high specificity. Further delineation as to specific target β-lactamases in a sample can be determined by use of β-lactamase inhibitors, as is further described herein.

A chromophore typically refers to a colorless or lightly colored chemical that an enzyme can convert into a deeply colored chemical that can be detected. In a particular embodiment, a chromogenic substrate is a substrate for a cysteine protease, as further disclosed herein. Once acted on by the enzyme (e.g., cysteine protease) the cleaved product can be quantified based upon measuring light absorbance at a certain wavelength, e.g., 350 nm, 375 nm, 380 nm, 385 nm, 395 nm, 400 nm, 405 nm, 410 nm, 415 nm, 420 nm 425 nm, 430 nm, 435 nm, 440 nm, 445 nm, 450 nm, 455 nm, 460 nm, 465 nm, 470 nm, 475 nm, 480 nm, 485 nm, 490 nm, 495 nm, 500 nm, 505 nm, 510 nm, 515 nm, 520 nm 525 nm, 530 nm, 535 nm, 540 nm, 545 nm, 550 nm, 555 nm, 560 nm, 565 nm, 570 nm, 575 nm, 580 nm, 585 nm, 590 nm, 595 nm, 600 nm, 650 nm, 700 nm 750 nm, 800 nm, 850 nm, 900 nm, 950 nm, or a range that includes or is in-between any two of the foregoing light absorbance values. For example, cleavage products for: Na-benzoyl-L-arginine 4-nitroanilide hydrochloride (BAPA) can be quantified by measuring light absorbance at 405 nm; L-pyroglutamyl-L-phenylalanyl-L-leucine-p-nitroanilide (PFLNA) can be quantified by measuring light absorbance at 410 nm; azocasein can be quantified by measuring light absorbance at 440 nm; pyroglutamyl-L-phenylalanyl-L-leucine-p-nitroanilide can be quantified by measuring light absorbance at 410 nm; resorufin can be quantified by measuring light absorbance at 570 nm; and unsymmetrical azo-based chromophores that can be quantified by measuring light absorbance at >520 nm. Any number of devices can be used to measure light absorption, including microplate readers, spectrophotometers, scanners, etc. In some instances, the detection can be observed by the naked eye and visualized as a colored “stripe” or “dot” etc. on a strip. The light absorption of the sample can be measured at various time points, e.g., 0 min, 1 min, 2 min, 3 min, 4 min, 5 min, 6 min, 7 min, 8 min, 9 min, 10 min, 11 min, 12 min, 13 min, 14 min, 15 min, 16 min, 17 min, 18 min, 19 min, 20 min, or a range that includes or is in-between any two of the foregoing time points. For example, the light absorption of the sample can be measured at 1 min and 5 min, or at various time points in between to establish a reaction rate.

Cysteine proteases, also known as thiol proteases, are enzymes that degrade proteins. These proteases share a common catalytic mechanism that involves a nucleophilic cysteine thiol in a catalytic triad or dyad. Cysteine proteases are commonly encountered in fruits including the papaya, pineapple, fig and kiwifruit. Caged or inactive cysteine proteases refers to cysteine proteases that can be activated by removal of an inhibitory segment or protein. For example, a caged/inactive papain would include papapin-S—SCH₃, whereby the inhibiting thiol segment can be removed by the breaking of the disulfide bond. Examples of cysteine proteases that can be used in the method disclosed herein, include, but are not limited to, papain, bromelain, cathepsin K, calpain, caspase-1, galactosidase, seperase, adenain, pyroglutamyl-peptidase I, sortase A, hepatitis C virus peptidase, sindbis virus-type nsP2 peptidase, dipeptidyl-peptidase VI, deSI-1 peptidase, TEV protease, amidophosphoribosyl transferase precursor, gamma-glutamyl hydrolase, hedgehog protein, and dmpA aminopeptidase. In a particular embodiment, a caged/inactive papain (e.g., papain-S—SCH₃) is used in the methods disclosed herein, in combination with a chromogenic substrate for papain (e.g., BAPA). Caged/inactive cysteine proteases can generally be reactivated by reacting with low molecular weight thiolate anions (e.g., benzenethiolate anions) or inorganic sulfides. In a particular embodiment, the targeting small molecule probes of the disclosure are a substrate for one or more targeted β-lactamases and release a benzenethiolate anion product:

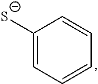

which then acts as a reaction amplifier by activating caged/inactive cysteine proteases (e.g., see FIG. 1).

For a method of the disclosure, the light absorbance of a sample can be compared with an experimentally determined threshold value to determine whether the targeted β-lactamase is present in the sample. For example, if the sample absorbance value is more than the experimentally determined threshold value, then the sample comprises a targeted β-lactamase. Alternatively, if the sample absorbance value is less than the experimentally determined threshold value or control value, then the sample likely does not comprise a targeted β-lactamase. Methods to generate an experimentally determined threshold value are taught in more detail herein, in the Examples section. Briefly, the experimentally determined threshold value can be determined by analysis of a receiver operating characteristic (ROC) curve generated from an isolate panel of bacteria that produce β-lactamases, wherein the one of more target β-lactamases have the lowest limit of detection (LOD) in the isolate panel.

The fabrication of the dry reagent test strips of the disclosure can be performed in a variety of manners. For example, the reagent test components can be combined or individually contained in a solution phase in reagent containers and then manually applied to the test pads of the dry reagent test strips. Alternatively, the reagent test components can be incorporated into an automated dispensing system, which then dispenses the reagent test components to the test pads of the dry reagent test strips. The application of the reagent test components to the test pads of the dry reagent test strips can be performed by lyophilization, stamping, chemical or physical entrapment, or coated onto the test pads.

In a particular embodiment, fabrication of the dry reagent test strips comprises the preparation of a coating solution. In one example, a primary coating solution is prepared using a mixture of the thiophenol-releasing small molecule (targeting small molecule probe) and an indicator (L-BAPA) in a low boiling point organic solvent such as methanol with a percentage of a stabilizing/excipient agent such as urea, sorbitol, trehalose, pullulan, polyethylene glycol, polyvinyl pyrrolidone, glycerol, sucrose, cellulose, dextran, or acacia gum. In another example, a secondary coating solution is comprised of a chemically modified papain in a buffer containing a percentage of a stabilizing/excipient agent such as urea, sorbitol, trehalose, pullulan, polyethylene glycol, polyvinyl pyrrolidone, glycerol, sucrose, cellulose, dextran, or acacia gum. In this secondary coating solution, the buffer composition can be 20-100 mM sodium phosphate or 20-100 mM sodium citrate/citrate buffer. The buffer composition may contain a percentage of ionic or non-ionic surfactants that may include, but is not limited to, triton X-100, tween-20 etc. In one embodiment, the primary coating solution is applied (10-50 uL) to a solid support. The solid support can be, but is not limited to a member of the group selected from plastic, nitrocellulose, cellulose, glass fiber, Teslin, filter paper etc. The plastic substrate can include, but is not limited to, high-density polyethylene (HDPE), low-density polyethylene (LDPE), polypropylene (PP), polyvinyl chloride (PVC), polycarbonate (PC), polysulfone (PSF), polystyrene (PS), Teflon (FEP), or Teflon (PFA). In one embodiment, the coated solid support with the primary coating solution is dried in a forced air oven with temperature ranging from 30-80° C. for a time needed to obtain drying. In another example, the solid support is dried under ambient conditions. In one embodiment, the secondary coating solution is applied to the solid support containing the dried primary coating and subjected to a second drying period. Further methods for fabricating the dry reagent test strips are provided in the Examples section herein. In some embodiments, the first and second coating solutions are applied at discrete locations on the substrate. In other embodiments, the first and second coating solutions are applied in layers up one another. In another embodiment, the coating solutions are applied at discrete locations such that upon use one location containing reagents is contacted with the sample prior to the second location, wherein the sample is “wicked” along the substrate bringing the contents of the two coating solutions/sites into contact.

The disclosure also provides for a kit which comprises one or more dry reagent test strips disclosed herein. A kit can further comprise one or more additional containers, each with one or more of various materials (such as reagents, controls, and/or devices) desirable from a commercial and user standpoint for use. Non-limiting examples of such materials include, but are not limited to, needles, syringes, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein. These other therapeutic agents may be used, for example, in the amounts indicated in the Physicians' Desk Reference (PDR) or as otherwise determined by one of ordinary skill in the art.

The following examples are intended to illustrate but not limit the disclosure. While they are typical of those that might be used, other procedures known to those skilled in the art may alternatively be used.

### EXAMPLES

Identification of the predominance of CTX-M type enzymes in Californian cUTI patient populations. Ceftriaxone non-susceptibility (minimum inhibitory concentrations [MIC] of ≥2 μg/mL) was used as a surrogate screen for ESBL-producing uropathogens. Simultaneously, the microbiology team performed a whole genome sequencing study which established that ceftriaxone resistant uropathogens predominantly harbored CTX-M type ESBL enzymes (identified in 91% of isolate collection) are primarily responsible for this phenotype circulating in cUTI patient populations within California, with the CTX-M-15 variant (56% of isolate collection) the most common CTX-M type enzyme identified.

Development of highly specific ESBL-targeting probes that specifically, and rapidly detect CTX-M type enzymes using a paper-based strip format. Due to the predominance of CTX-M type enzymes in cUTI patients, ESBL-targeting probes that specifically detect CTX-M-15 and other common CTX-M-type variants were focused on. A first ESBL-targeting probe that specifically detected CTX-M-type was identified. This ESBL-targeting probe, (7R)-8-oxo-7-(2-phenylacetamido)-3-((phenylthio)methyl)-5-thia-1-azabicyclo[4.2.0]oct-2-ene-2-carboxylic acid (Sub-1), was used as an internal reference standard in establishing acceptable thresholds of detection limits and targeting probe performance in clinical urine samples. While the performance metrics of Sub-1 were adequate for clinical use, the stability and promiscuity characteristics of Sub-1 were not adequate for rapidly detecting ceftriaxone non-susceptibility directly from urine.

In order to find use in rapid diagnostic tests, ESBL-targeting probes that had at least the following favorable criteria were evaluated:
1. Aqueous stability—measured through non-selective hydrolysis
   a. Favorable criteria: k(hydro)<0.0062 mM/min
2. Activity towards target β-lactamase target CTX-M-15—measured using recombinant β-lactamase
   a. Favorable criteria: k(β-lactamase)>0.0568 mM/min
3. Activity towards off-target lower generation β-lactamase (prioritizing the most common, TEM-1b)
   a. Favorable criteria: k(TEM-1b)<0.02135 mM/min

ESBL-targeting probes that exhibited the above favorable criteria were further investigated to determine the kinetic parameters and preference for CTX-M-15 (target) over TEM-1b (off-target) to minimize the potential of detection of false positives. ESBL-targeting probes that demonstrated ˜10-fold improvement in specificity towards CTX-M-15 (common target enzyme) over TEM-1b (common off-target enzyme) were determined to be strong candidates for further testing with a comprehensive panel of 96-bacterial isolates, to establish the capacity of each targeting probe to resolve target and off-target β-lactamases. Further, the ESBL-targeting probes were evaluated using a paper-based strip format.

A pool of 5 probe hits were each evaluated using a 96-bacterial isolate panel. In these studies, the 96-bacterial isolate panel was comprised of 47 cefotaxime (CTX) susceptible and 49 CTX non-susceptible, Gram-negative bacteria (GNB). As described above, CTX is a surrogate to detect ceftriaxone susceptibility and, therefore, was applied as the reference phenotype to group true positive, CTX-non-susceptible (include both CTX resistant and intermediate), and true negative, CTX-susceptible. Whole cell suspensions were prepared to yield an experimental concentration of 2.5×10⁶ CFU/mL. In a 96-well plate, wells were prepared with all DETECT reagents, except for the targeting probe under investigation. Bacterial suspensions were transferred to the prepared wells of the plate and an initial timepoint was recorded (0 minutes) upon addition of a stock solution of a given probe (Sub-1, (7R)-7-(2-(1H-tetrazol-1-yl)acetamido)-8-oxo-3-((phenylthio)methyl)-5-thia-1-azabicyclo[4.2.0]oct-2-ene-2-carboxylic acid (BAD-5835), (7R)-7-(3-methylisoxazole-5-carboxamido)-8-oxo-3-((phenylthio)methyl)-5-thia-1-azabicyclo[4.2.0]oct-2-ene-2-carboxylic acid (BAD-1468), (7R)-8-oxo-3-((phenylthio)methyl)-7-(2-(pyridin-3-yl)acetamido)-5-thia-1-azabicyclo[4.2.0]oct-2-ene-2-carboxylic acid (BAD-5627), and (7R)-8-oxo-3-((phenylthio)methyl)-7-((S)-piperidine-3-carboxamido)-5-thia-1-azabicyclo[4.2.0]oct-2-ene-2-carboxylic acid (BAD-3462), (7R)-8-oxo-7-(2-phenoxyacetamido)-3-((phenylthio)methyl)-5-thia-1-azabicyclo[4.2.0]oct-2-ene-2-carboxylic acid (BAD-7465)). The absorbance of the wells was collected at 405 nm at 0 and 30 minutes, using a simple benchtop plate reader. The “DETECT Score” values were then calculated for each sample by subtracting the 405 nm absorbance recorded at the 0-minute timepoint from the 30-minute timepoint. Therefore, each sample was identified to have a unique DETECT score that was then can be group into positive and negative phenotype subgroups for threshold value determination via ROC curve analysis.

The criteria applied to define two lead probes from the 5 hits included (1) general stability as a measure of the rate of non-selective hydrolysis of the a given probe (lower k(hydro) the more stable the probe); (2) the profile of “true positive” (TP) from “true negative” (TN) detected by each of the probes, and (3) overall accuracy as defined by applying a standard receiver operator characteristic (ROC) curve.

An overview of the results of these studies are denoted in Table 2. Targeting probes BAD-5835 (G2B) and BAD-5627 were determined to be strong lead candidates due to their stability, high sensitivity and specificity.

TABLE 2
Summary of the key results collected for all 5 hit probes to establish
the 2 lead candidates. Stability of each probe is reported in
k(hydro), units of molarity per min, which corresponds to the
rate of non-selective hydrolysis of the probe and turn on of
the DETECT system in the absence of its biomarker target.

| | Stability | ROC Curve Analyzed with CTX | | |
|---|---|---|---|---|
| | k(hydro) | | | AUC |
| BAD-Probe | M/min | Sensitivity | Specificity | (95% CI) |
| Sub-1 | 6.20 × 10⁻³ | 0.9206 | 0.8125 | 0.7717 |
| G2B | 4.40 × 10⁻³ | 0.8823 | 0.9259 | 0.9586 |
| BAD-5627 | 3.52 × 10⁻³ | 0.9275 | 0.9259 | 0.9614 |
| BAD-1468 | 1.98 × 10⁻³ | 0.8406 | 0.8519 | 0.8846 |
| BAD-3462 | 5.58 × 10⁻³ | 0.8116 | 0.8519 | 0.8717 |
| BAD-7465 | 1.61 × 10⁻³ | 0.7143 | 0.8077 | 0.7717 |

Evaluation of BAD-5835 and BAD-5627 ESBL-targeting probes using a fully characterized, uropathogen isolate test set. BAD-5835 and BAD-5627 are evaluated using a fully characterized, uropathogen isolate test set which comprises uropathogens collected at San Francisco (SF) General Hospital in SF, CA in 2019 (n=650). These isolates were phenotypically characterized and are currently being genotypically characterized by whole genome sequencing (in partnership with Illumina). This test set was chosen because the uropathogens were cultured from urine samples sent to the central lab for suspected urinary tract infection (UTI) at the public hospital. Therefore, it is representative of the patient population that is evaluated with the rapid diagnostic solid-phase strip test format (e.g., U-detect) disclosed herein. Patient data will not be known and only organism information will be provided in order to establish the test performance metrics using BAD-5835 or BAD-5627 in a rapid diagnostic solid-phase strip test format (e.g., U-detect) of the disclosure.

Decreasing time-to-results of a chemistry-powered detection system to yield results in less than five minutes to reach a target product profile. To allow for immediate guidance on antibiotic prescribing, acceleration of the time-to-result dynamics were evaluated. Currently, there are no rapid diagnostics on the market that can guide the initial antibiotic therapy selection for the treatment of complicated urinary tract infections (cUTIs). Bacterial culture and antibiotic susceptibility testing (AST) are the current gold standard, however, it's TTR can take up to 3 days. Solution based DETECTs (i.e., DETECT 1.0) TTR is currently 30 minutes, including assay incubation time, to yield antibiotic susceptibility results directly from the patient's urine sample. It has become evident that the ideal target product profile (TPP) for a rapid diagnostic includes a TTR with a 5-minute timeframe. The product format is a urine dipstick test (i.e., a dry reagent strip test), which is part of the current diagnostic workflow for the treatment of cUTIs. The urine dipstick test's TTR is five minutes, which makes it extremely effective in the diagnostic workflow as it immediately provides results which can support selection of appropriate antibiotic therapy. In addition to this, it is a part of the clinician's workflow, which lowers the implementation barrier into the hospital setting. Thus, for U-detect to be clinically effective, it is important that it meets the current urine dipstick test's TTR of five minutes. Therefore, to achieve true rapid and clinically effective TTR of five minutes or less the signaling tier of DETECT 1.0. requires improvement.

DETECT 1.0 is comprised of two tiers: targeting and signaling. The signaling tier of DETECT is responsible for providing the signal output when chromogenic BAPA is hydrolyzed by papain. The rate of signal output by the DETECT 1.0 system is dependent on the rate of hydrolysis of BAPA by papain. Thus, to achieve an optimal TTR of five minutes or less for U-detect, the kinetic rate BAPA hydrolysis needs to be increased. To achieve this, the current compound, BAPA, will be modified to create a library of BAPA derivates with improved rates of hydrolysis (k_{cat}, k_{cat}/Kₘ), which will translate to faster signal output and thus decreased TTR BAPA derivatives comprising modifications of a highly conserved sequence of a hexapeptide (X-QVVAGA-Y, X & Y=quencher and dye) that demonstrated dramatic substrate specificity (at 30° C., kcat/Km 2.9×10⁹ M⁻¹ min⁻¹) but suffered from solubility issues were investigated. To improve solubility the peptide the peptide will be modified to incorporate hydrophilic amino acid residues. The resulting BAPA derivatives will be examined for improved rate of hydrolysis at ambient temperature.

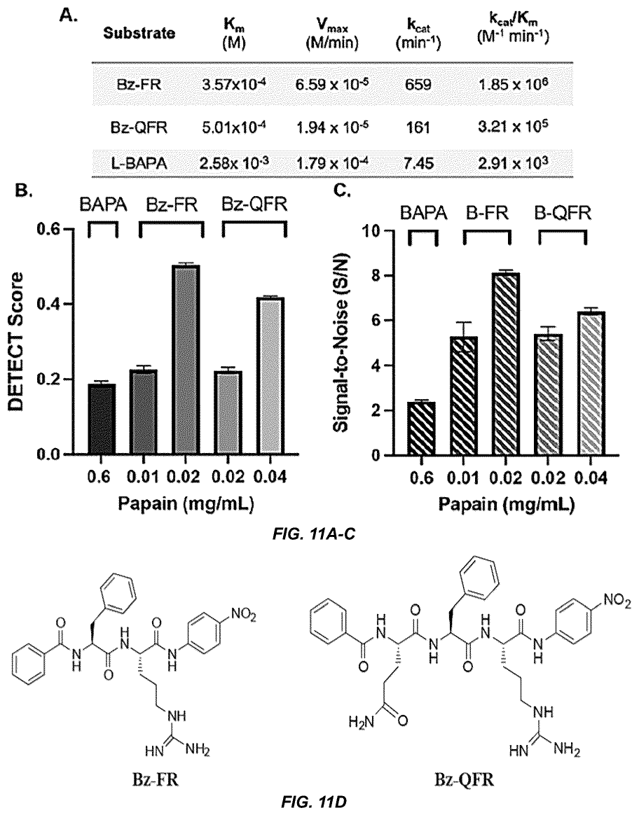

Establishing feasibility of improving time-to-results (TTR) of DETECT by examining improved rate of BAPA hydrolysis. The BAPA (Bz-R-pNA) substrate utilizes an arginine (R) residue to direct the modest recognition by papain. However, because it has been established that papain uses hydrophobic residues, a BAPA derivative that incorporated a phenylalanine residue (F) and a glutamine residue (Q), named Bz-FR and Bz-QFR substrates, respectively, were first evaluated. Both peptides contained the N-terminal benzoyl (Bz) & the C-terminal chromogenic paranitroanilide (pNA) of BAPA. The hydrolytic rates of the Bz-FR & Bz-QFR substrates were compared to BAPA using DETECT 1.0. Additionally, the ability of the tri- and dipeptide substrates to provide significant absorbance change (DETECT score) and maintain high signal-to-noise (S/N) at 5 minutes were examined (FIG. 11). Introduction of the Phe residue provided a dramatic increase in hydrolysis relative to BAPA, with ˜7-fold improvement in binding affinity (Kₘ) and 88-fold increase in turnover number (k_{cat}), which resulted in 1000-fold increase in substrate specificity (k_{cat}/Kₘ) for the Bz-FR substrate as shown in FIG. 11A. Similarly, the tripeptide Bz-QFR that incorporated a Gln residue did not appear to be as active as the Bz-FR substrate, but provided modest improvement over the BAPA. Overall, both alternative BAPA derivates provided an improvement in absorbance change (defined as the DETECT Score) (see FIG. 11B) and an increase in S/N (see FIG. 11C) upon papain activation. Moreover, the assay used at least 60× less papain than what BAPA (DETECT 1.0) utilizes.

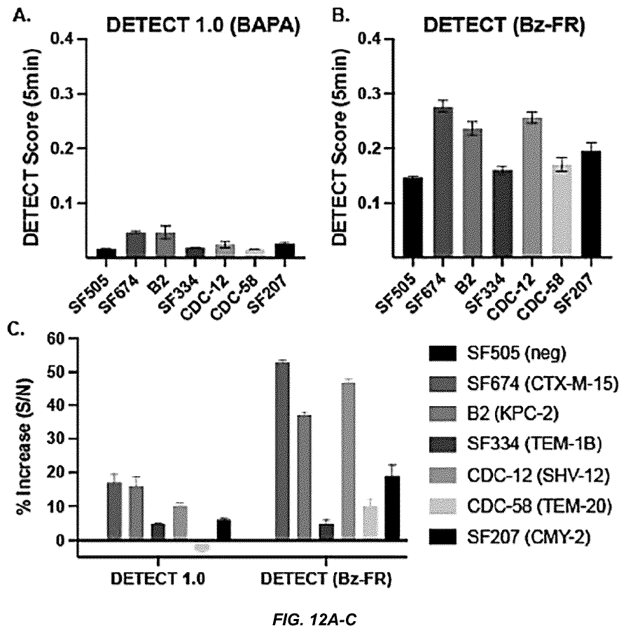

Demonstrate improved time-to results (TTR) with alternative papain substrate Bz-FR in clinical isolates. As the Bz-FR substrate possessed superior rate of hydrolysis and maintained high S/N in the papain activation study, Bz-FR was then examined for its ability to yield optimal signal output in 5 minutes with clinical bacterial isolates. This study utilized the ESBL targeting probe (BAD-5835) to detect cefotaxime coupled with the Bz-FR papain substrate, to yield the signal output. Clinical isolates that are resistant to cefotaxime and produced β-lactamase enzymes, CTX-M-15 (SF674), SHV-12 (CDC-12), CMY-2 (SF207), KPC-2 (B2) & TEM-20 (CDC-58), hereby considered as “positive” isolates, were examined against “negative” isolates (SF334 & SF505) which were susceptible to cefotaxime. At a standard concentration of 10⁶ CFU/mL for each isolate, the DETECT scores were examined after 5 minutes of assay incubation for DETECT 1.0 and DETECT with Bz-FR Additionally, the S/N was examined between each assay condition to assess how the signal from ‘positive’ resistant isolates compared to the signal generated by ‘negative’ isolates. For DETECT 1.0, the signal that was generated at 5 minutes was difficult to distinguish between the positive and negative isolates, with SF674 and B2 producing a signal greater than the negative isolate (˜3-fold higher) (see FIG. 12A). For the Bz-FR containing DETECT, there was a much higher change in absorbance after 5 minutes across the positive and negative isolates but had only a ˜2-fold increase in signal when comparing the positive (SF674) to the negative (SF505) isolate (see FIG. 12B). However, for BZ-FR, all positive isolates produced a DETECT signal greater than the negative controls, ranging from up to 1.9-fold greater than the average signal for the negative controls. Additionally, there was a ˜50% in increase in signal for the positive (SF674) compared to the negative (SF5050) for the Bz-FR (see FIG. 12C). Continuing to improve on the level of background signal that is generated from a negative isolate (or true negative sample) will be useful to having a robust test that can discern true positive (antibiotic resistant) from true negative samples (antibiotic susceptible). Ways to minimize the background signal that was generated with the Bz-FR in the negative isolates needs further improvement.

The focus of this objective is to identify a signaling substrate for papain that can maintain a high level of signal in true positives (ceftriaxone-resistant) while reducing the amount of signal that is generated in negative isolates (ceftriaxone-susceptible). This will be accomplished by examining derivatives of the Bz-FR substrate by conducting incremental changes to this scaffold by first replacing the recognition residue at the 2^{nd} position such as the (F) residue with alternative hydrophobic residues to dampen the background noise that can be generated in isolates. Secondly, the (F) and (R) positioning will be swapped to assess whether the over reactivity can be dampened. Each Bz-FR derivative will undergo rigorous evaluation using a pipeline of validation where certain criteria must be met to ensure an optimal candidate can be identified and used in objective 2 and ultimately in the test form. In view thereof, four BAPA derivatives will be evaluated which will include substituting for the Phe residue with Val and Trp, and separately the Arg residue will be replaced with Lys and the Arg and Phe will be exchanged for one another. The BAPA derivatives will be tested using an activity assay that uses cysteine for papain activation to determine kinetic parameters, and establish a ratio of papain to the BAPA derivatives to maximize S/N. Those BAPA derivatives with S/N>3 and DETECT score>0.25 will be used for further optimization using a recombinant target biomarker, CTX-M-15, and the targeting probe BAD-5835, with an assay incubation time of 5 minutes. From which, an optimal ratio of papain to a BAPA derivative can be identified, and the level of noise generated from BAD-5835 from spontaneous hydrolysis can be determined.

The BAPA derivates that passed the biochemical testing phase will be subjected to microbiological testing (stages 3-7) using clinical isolates. In stage 3, each BAPA derivative will be examined for its ability to evade hydrolysis by bacterial proteases or amidases. If there is no indication that the BAPA derivative was non-specifically hydrolyzed, in stage 4 it will be tested for its ability to produce a viable signal for positive isolates, containing β-lactamase target biomarkers, which can be discerned from negative isolates, in 5 min or less. For a BAPA derivative to be considered for further testing it should demonstrate a DETECT score 0.25 or greater for the positive control isolate, and for the negative control isolate (SF505), to demonstrate a DETECT score<0.15. If the level of signal output is achieved, in stage 5 the BAPA derivative will be further optimized by conducting higher resolution of the ratio of BAPA derivative to papain through optimization using a checkerboard assay to maximize signal output. With significant improvements to the signal output, a limit of detection (LOD) study will be carried out using a 7-clinical isolate panel to demonstrate an LOD of less than 10⁵ CFU/mL. Lastly, optimized BAPA derivative with targeting probe BAD-5835 will be tested against a 96-bacterial isolates panel that is comprised of 47 cefotaxime susceptible and 49 cefotaxime resistant, gram-negative bacteria. A DETECT score is calculated for each isolate sample and is grouped into the positive and negative phenotype subgroup to establish a threshold value. Clinical isolates that generate a DETECT score that is greater than the threshold value will be considered positive, and the sensitivity and specificity of the DETECT containing the BAPA derivative will be determined by analyzing DETECT signals via a receiver operating characteristic (ROC) curve analysis. Ideally for the U-DETECT assay, the BAPA derivative will generate 90% sensitivity and 92% specificity in 5 minutes.

Tuning of the signaling output of a chemistry-powered detection system from 405 nm to 525 nm for universal compatibility with urine analyzer systems. While having a rapid TTR will be advantageous, maximizing the sensitivity and tuning of the signal wavelength of DETECT 1.0 will allow better positioning of the technology for multiple applications such as, point-of-care testing, visual detection, and multiplexing. Adaption of the DETECT technology will also ensure compatibility of this test with existing urine analyzers. As a dry reagent test strip (U-DETECT) that can be fitted onto existing urine analyzers, it is imperative that the optical specifications (in particular, the wavelength of the signal output) of U-DETECT is compatible with existing analyzers on the market to reduce the barrier of adoption of the test. Replacement of the current signaling output moiety (p-nitroaniline) will be a critical step to matching the signaling wavelength to those on urine analyzer systems such as Beckman Coulter's iChemVelocity & Siemen's CLINITEK Novus that, which have the largest market share.

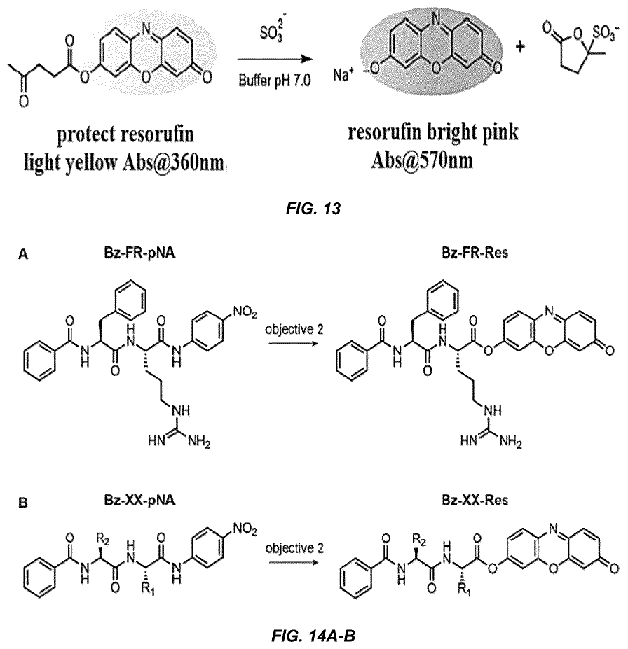

The signal output of DETECT 1.0 is currently read at 405 nm, and through modification of the signaling output molecule, a longer wavelength (525 nm or greater) can be obtained with improved photophysical properties that enables sensitive detection of ceftriaxone resistance. In the signaling tier of DETECT 1.0, BAPA is hydrolyzed by papain to liberate the chromogenic p-nitroaniline upon triggering of the signaling cascade from the presence of an ESBL target biomarker. This chromogenic moiety has been widely used in substrates for proteolytic enzymes but has several drawbacks that would allow it to be considered in diagnostics. The p-nitroaniline is a relatively poor chromophore with an extinction coefficient (E) of 9,767 M-1 cm⁻¹ at its absorbance wavelength of 405 nm. Given that the intensity of absorption is proportional to the extinction coefficient, replacement of the p-nitroaniline chromophore moiety with a chromophore that has an extinction coefficient of >25,000 M⁻¹ cm⁻¹ and absorbance wavelength of >525 nm would be suitable. One viable candidate is the fluorescent indicator has been widely used as a chromophore, resorufin, which has an (E) of 56,000 M¹ cm⁻¹ at its absorbance wavelength of 570 nm. Additionally, it has a relatively low pka (OH group), high water solubility (1 mg/mL) and high fluorescent quantum yield (ϕ=0.74 in water). Due to resorufin's high extinction coefficient, the color change converts from light yellow (quenched form) to vibrant pink (unquenched) which can be observed by the naked eye. One notable application of resorufin, was as a chromogenic probe for the detection of the anion sulfite (SO₃²⁻) that was demonstrated by Choi and coworkers. The resorufin was quenched (“turned-off”) by addition of a protecting group that is susceptible to cleavage by the presence sulfite. Once the protected resorufin encounters sulfite, the protecting group will be cleaved to yield the chromogenic resorufin with a 320-fold response in absorbance change (see FIG. 13). Additionally, this chromogenic probe acts dually as a fluorescent molecule and its structural changes can be monitored by fluorescence where the sulfite probe provided a 57-fold increase in fluorescence with a limit of detection of 4.9×10⁻⁵ M for sulfite. Therefore, resorufin is a viable candidate to investigate as the next generation signaling output for DETECT to enable adoption and testing with existing urine analyzers.

Replacement of the chromogenic p-nitroaniline with commercial chromophores to improve sensitivity and increase the absorbance wavelength to >525 num. Using the current candidate Bz-FR-pNA, the p-nitroaniline (pNA) chromophore will be replaced with resorufin (Res) to yield the Bz-FR-Res signaling output molecule (see FIG. 14A). Additionally, BAPA derivatives will also undergo investigation with the resorufin chromophore (see FIG. 14B). The generated BAPA-Res derivatives (dBAPA-Res) will undergo stringent biochemical and microbiological validation, using the approach that is described above. An expedited validation pipeline will be applied to identify a dBAPA-Res candidate with improved rate of hydrolysis to yield the signaling output at 5 minutes or less, improved sensitivity for the target biomarker, and have a longer wavelength (>525 nm) of absorbance for compatibility with existing urine analyzers (see FIG. 14B).

Synthesis and development of a chromogenic moiety for DETECT to improve sensitivity and increase the absorbance wavelength to greater than 525 nm. Given the ease of synthesis and ability to chemically tune azo dyes its absorption wavelengths ranging from 400 nm and up to 600 nm with extinction coefficients as high as 50,000 M⁻¹ cm⁻¹, azo-based chromophores fit those required of the target product profile. Motivated by the photophysical properties of azo-based chromophores, an azo-based chromogenic moiety for DETECT that will have improved sensitivity (over DETECT 1.0 with BAPA) and be compatible with existing urine analyzers will be developed.

Synthesis and development of a novel chromogenic dBAPA-Azo derivative. A novel chromophore will be identified with an absorbance wavelength greater than 525 nm. This will be achieved by synthesizing an unsymmetrical azo-based chromophore that incorporates the benzothiazole moiety tethered to an aniline group via the diazo functionality (see FIG. 15A). The benzothiazole acts as an electron withdrawing group from the aniline to facilitate the intramolecular charge transfer (ICT) process. This chromophore will be obtained by the diazotization two-step coupling reaction. The benzothiazole amine (1) will undergo diazo formation to give the intermediate (2). Molecule (2) then undergoes an electrophilic aromatic substitution with the aniline derivative (3) to furnish the desired chromophore (4). To ensure that a chromophore with superior properties over p-nitroaniline is identified, twelve azo-based derivatives with diverse functionalities will be evaluated. There will be 4 representative scaffolds (X═CN, NO₂, Br, Cl) that will be comprised of functionalities that promote the ICT mechanism for longer wavelengths and high extinction coefficients. Within each of these scaffolds, various R groups (H, OCH₃, or SO₃) will be examined for improved photophysical (wavelength & extinction coefficients) and physical (solubility) properties. Due to the hydrophobic nature of these chromophores, the photophysical properties will be first evaluated in organic solvent such as dichloromethane, methanol, and acetonitrile. This allows characterization of the chromophore in different polarities and assess how the absorbance changes in these conditions. Next, the solubility in the water and the buffer condition that DETECT operates in will be evaluated to monitor the absorbance with respect to concentration. Lastly, the absorbance in acidic (pH 4), neutral (pH 7), and basic (pH 9) conditions will be examined to establish that pH does not have a negative effect on absorbance (i.e., decrease in acidic or neutral). A chromophore derivate that exhibits wavelengths greater than 525 nm with extinction coefficients greater than 25,000 M⁻¹ cm⁻¹, and have water (and/or buffer) solubility ranging of between 0.25-0.5 mg/mL will be considered as a lead to perform the C-terminal modification to each peptide backbone that was identified.

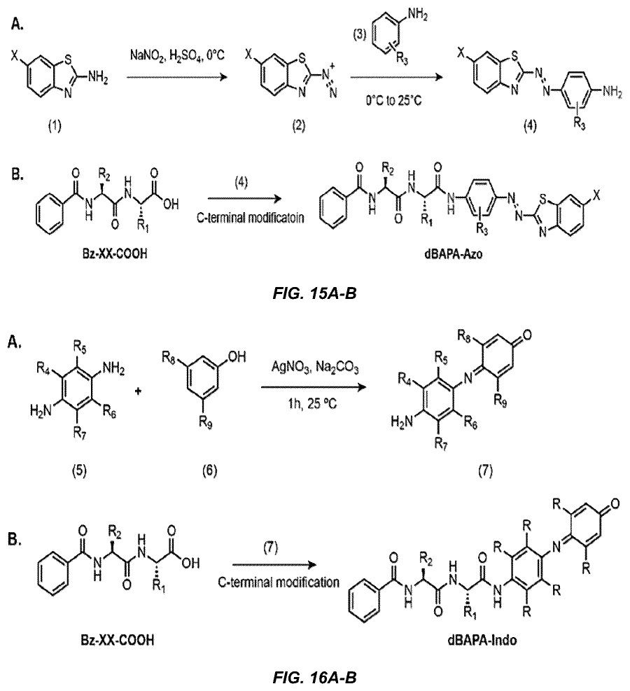

Synthesis of an indoaniline chromogenic moiety for DETECT to improve sensitivity and increase absorbance wavelength to >525 num. Indoaniline chromophores are derived from an aniline and a phenolic compound where in the presence of an oxidant will provide the indoaniline product. The indoaniline class of chromophores have been widely used in the permanent hair coloring industry with patents for the chromophore components dating back to as early as 1880s. The tunability of these chromophores have enabled chemists to develop a variety of colors with diverse physical and photophysical properties that can range from absorbance wavelengths from 500 and up to 700 nm with extinction coefficients greater than 25,000 M⁻¹ cm⁻¹. Due to their ease of synthesis and tunability, these classes of chromogenic dyes are well suited for incorporating into the signaling tier of DETECT to improve sensitivity and increase the signaling wavelength (see FIG. 16A).

Synthesis, and development of a chromogenic indoaniline derivative to yield a dBAPA-Indo papain substrate. Nine indoaniline derivatives will be investigated to identify a candidate with absorbance wavelength greater than 525 nm. The scope of work will consist of synthesis and structural characterization, and physical and photophysical characterization such as solubility and absorbance measurements in different conditions. Lastly, the coupling efficiency studies will be carried out to ensure a level of reactivity for conjugation onto the peptidyl papain substrate is present. Upon identifying a lead chromogenic indoaniline derivative, this component will be modified on the C-terminal (see FIG. 16B).

Validation of chromogenic dBAPA derivatives to demonstrate improved sensitivity and increased absorbance wavelength for interpretation by existing analyzers. Although the backbone (Bz-XX & Bz-FR) of the papain substrate has been validated to demonstrate improved TTR, the chromogenic substrates will be subjected to a validation pipeline to maintain the rate of hydrolysis for rapid TTR and demonstrate improved sensitivity for detecting the target biomarker. Biochemical validation of dBAPA-Res, dBAPAAzo, dBAPA-Indo derivatives will be carried out using the papain activation assay. In the papain activation assay (stage 1), assessment of the S/N and absorbance change (DETECT score) is maintained at 5 minutes or less will be determined relative to the lead p-nitroaniline containing substrates (Bz-XX-pNA & Bz-FR-pNA). Additionally, the catalytic efficiency will be investigated to ensure the rate of hydrolysis was not drastically affected by the introduction of each chromophore derivative, and the kcat/Km is maintained in the order 10⁵-10⁶ M⁻¹ min⁻¹. Three top candidates that could maintain (or improve) rate of hydrolysis, S/N, and DETECT score at 5 minutes or less will be identified.

Microbiological validation of dBAPA-Res, dBAPA-Azo, and dBAPA-Indo derivatives will be carried out against a panel of clinical, E. coli isolates. This panel consists of 7 isolates that were obtained from patient blood or urine samples and exhibit various sensitivities to cefotaxime. In stage 2, each BAPA chromophore derivative will be examined for its ability to evade hydrolysis by bacterial proteases or amidases, and to ensure that the incorporation of the chromophore does not promote non-specific hydrolysis. In stage 3, an LOD study using the clinical isolate panel will be conducted to demonstrate an improved detection limit of <10⁵ CFU/mL for cefotaxime resistant isolates and ensure that there is insignificant signal from true negative isolates (sensitive to the action of cefotaxime). If needed, optimization of the BAPA derivative to papain ratio can be conducted (stage 4) to maximize signal output. This will only be considered if significant noise is generated from non-specific hydrolysis of the targeting probe (BAD-5835). Lastly, in stage 5, a verification study using a 96-isolate panel will be conducted to ensure sensitivity and specificity is maintained relative to DETECT 1.0. A DETECT score that is greater than the threshold value will be considered positive, and the sensitivity and specificity of the modified assay will be determined by analyzing DETECT signals via a receiver operating characteristic (ROC) curve analysis. The chromogenic BAPA derivative that can produce at least 90% sensitivity and 92% specificity in less than 5 minutes will be identified as the lead chromogenic substrate for papain, which will be used for further development for U-DETECT.

Feasibility of translating assay components onto strip format by demonstrating activity of components is retained (relative to assay) by cysteine activation. Preliminary feasibility studies for the dry regent test strips will focus on the following 5 criteria:
(1) Solid phase material evaluation, comparison (resistance to dry out over 30 min) with saturated sample volume, identify suitable test strip configuration to retain moisture over time.
(2) Successful dry down studies on solid phase materials: cellulose and glass fiber (dry time and temperature).
(3) Assay read time evaluation (20 minutes) on solid phase materials per sample type (dosed on paper and dried on paper).
(4) Very minimal formulation optimization to enhance signal produced on selected membrane(s).
(5) Sample type comparison: spiked aqueous samples (cysteine solution, 10 μL) compared to assay format (evaluation at 405 nm).

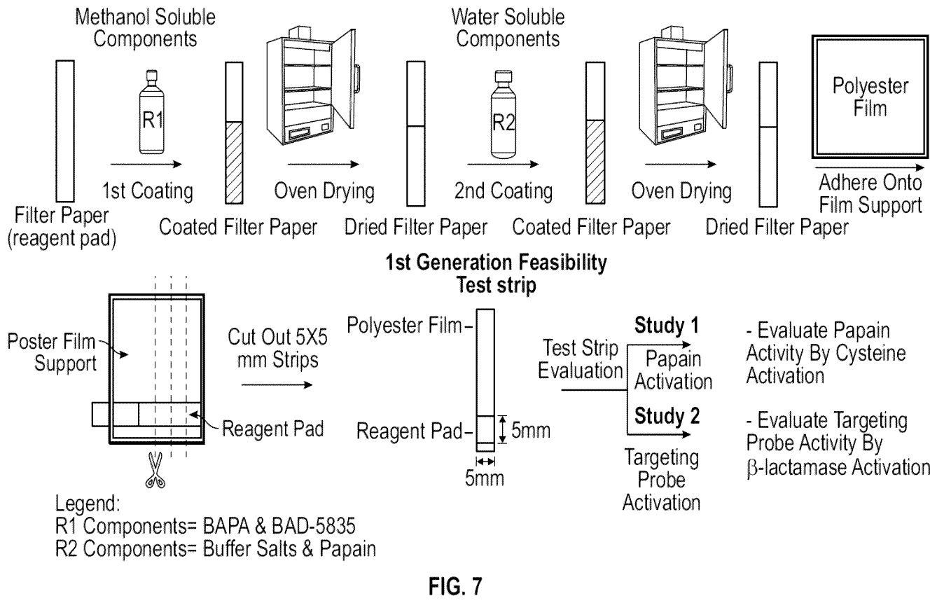

Overview of Fabrication process: Using a grade-222 filter paper as the solid matrix (reagent pad), the DETECT assay components were coated in two steps. The first step consisted of coating the reagent pad with methanol soluble components (BAPA & BAD-5835) and dried in a forced air oven set at 37° C. The second coating consisted of adding the water-soluble components (buffer salts and papain) and dried a second time in the oven at 37° C. The reagent pad is then adhered onto a polyester film and then cut into 5×5 mm reagent strips to undergo testing (see FIG. 7). Multiple formulation combinations of the DETECT components were examined to determine optimal change of reflectance over time.

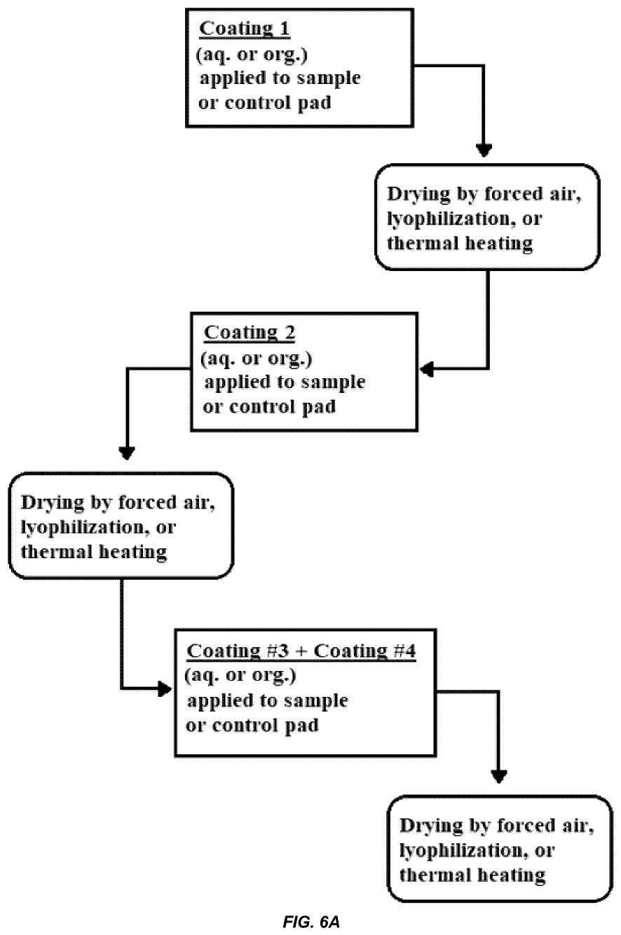

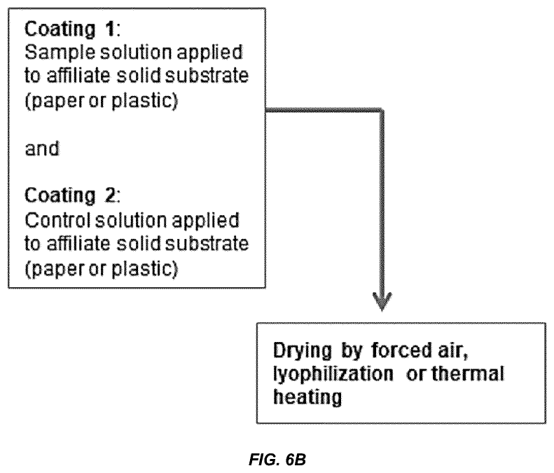

Fabrication of test strips: An overview of the fabrication process from liquid phase to solid phase test is shown in FIG. 6. The fabrication process starts by cutting a piece of the selected reagent pad (Ahlstrom-Munksjö Grade 222 filter paper). To conduct the first coating step, a solution of the methanol soluble components (BAPA & BAD-5835) is transferred to a weigh boat and the filter paper is fully submerged to ensure full saturation. Any excess solution is removed with glass rods and is placed in a forced air oven and dried for 45 minutes at 37° C. The second coating is carried out by preparing the water-soluble components (10 mM sodium phosphate buffer, 25 mM Bis-tris, 1.125 mM EDTA, 12.5 mM NaCl, and papain at 0.6 mg/mL) and transferring the solution to a weigh boat where the dried filter paper can be fully submerged to ensure saturation. Excess solution is removed with glass rods and the paper placed in the oven and dried for 60 minutes at 37° C. In a dry room (relative humidity<20%), the dried filter paper is adhered onto a polyester film support. Using a 3M 415 double sided film tape, the dried filter paper is adhered onto a piece of Tekra 10 mil MELINEX® 339 opaque white polyester film and light pressure is applied with a hand roller to ensure the filter paper has adhered. The filter paper adhered to the film is then cut into 5 mm wide strips to provide the test strip with a 5×5 mm reagent pad (FIG. 1). The order of applying the DETECT components typically includes adding the methanol soluble components first followed by the water-soluble components. Having methanol as the second coating step can be detrimental to papain. Additionally, it was established that at least 20 IL of sample volume was effective to prevent drying out of the reagent pad during the incubation time of 20 minutes.

Feasibility of dry reagent test strips for detecting antibiotic resistant bacteria. Papain activity by cysteine activation was assessed by using two test strips where one received 100 μM cysteine (sample) while the other received buffer (control). For each strip the reflectance was monitored by percent reflectance (control subtracted) over time.
Study 1 materials: Papain, BAPA, BAD-5835 and cysteine (100 μM).
Study 2 materials: Papain, BAPA, BAD-5835 and CTX-M-15 (1 nM or 4 nM).

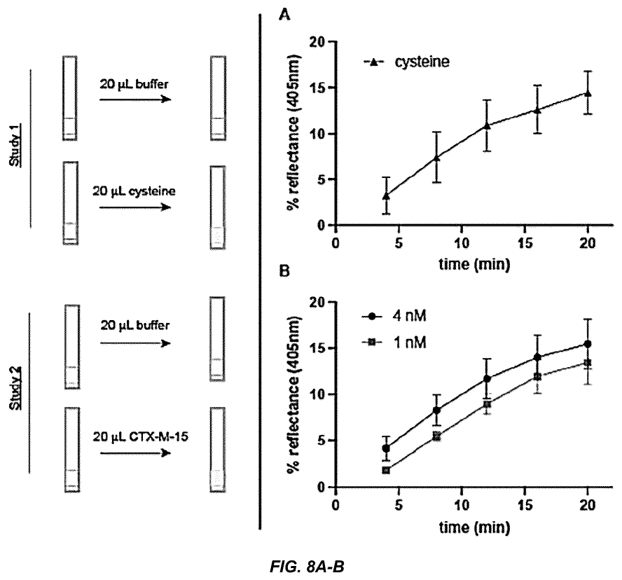

To demonstrate the DETECT components sustained the fabrication process, two separate studies were carried out where in study 1. papain activity was evaluated by cysteine activation and in study 2. The β-lactamase targeting probe was evaluated by CTX-M-15 activation. In study 1, different formulations (i.e., different amounts of the papain and BAPA) were used to prepare strips to obtain the highest possible reflectance change when the yellow-colored signal is generated from activating papain (se FIG. 8) and measured with a Konica Minolta reflectance spectrophotometer. With formulation 1 as the baseline concentration, which used 0.6 mg/mL (1×) of papain, 2.56 mg/mL (1×) of BAPA, and 1.28 mg/mL (1×) of BAD-5835. Any formulation that was used hereafter, was either an increase in the amounts of all the components or just papain & BAPA.

Study 1. Single reagent pad test strips were prepared to investigate how best to address reflectance upon activating the signaling cascade and optimization of the signaling. Initially, four unique formulations (1-4) were used where either the papain or simultaneously all components were increased with respect to formulation 1 (1×). For each formulation, the ability to activate papain by cysteine was examined. For each formulation, two test strips were dedicated for an experiment run where one test strip received 20 μL of 100 μM cysteine (sample) while the other test strip received 20 μL sodium phosphate buffer (control) and the percent reflectance (using 405 nm wavelength) were recorded every 4 minutes until 20 minutes of incubation time at ambient temperature had elapsed (see FIG. 8). In the absence of color, most of the light is reflected, while in the presence of color, significant amounts of light are absorbed, and the amount of light reflected is decreased. Therefore, as color signal is generated from the DETECT system, there is a decrease in percent reflectance over time. The difference of percent reflectance (% Rscore) between sample and control is determined by subtracting the percent reflectance of the control strip from the sample strip for each time point to yield an increasing % Rscore over time (see FIG. 9A-B). Similarly, the change of reflectance over time to yield the DETECT % Rscore (% Rscore_{20 min}−% Rscore_{2 min}) was determined to demonstrate which formulation observed the highest change after 20 minutes of incubation time.

Study 1.1 & 1.2 Assessment of DETECT components on the test strip by cysteine activation of papain using unique formulations. Study 1.1, a DETECT % Rscore was observed with as high as 7.70 and 7.73 for formulations 2 & 4, respectively. Given that formulation 4 used higher amounts of the BAD-5835 targeting probe and is usually a contributor to noise (in assay form at least), formulation 2 was pursued as the focal point for optimizing the conditions where the papain and BAPA amounts was increased while keeping the BAD probe constant.

Study 1.2, as high as four times the formulation 1 (with respect to papain and BAPA) was examined for maximizing the increase of % Rscore for papain activation by cysteine. Increasing the amount papain and BAPA was able to provide an improved % Rscore as shown in FIG. 9D relative to formulation 2 where formulation 7 and 9 provided similar responses over time. Additionally, formulations 7 and 9 provided similar improvements based on the DETECT % Rscore, which was the highest observed thus far in these feasibility studies. This is an example where papain activation by cysteine is demonstrated in the test strip format. This example shows that the DETECT components can be translated to the test strip format.

Results of Study 1. Papain was able to sustain the fabrication process and retain its enzymatic activity as demonstrated by cysteine activation. The BAD-5835 targeting probe did NOT decompose from the fabrication process, which the control strip would have shown significant change in reflectance if it had degraded. Increasing the amount of papain in the test strip gave an overall increase in % Rscore and DETECT % Rscore.

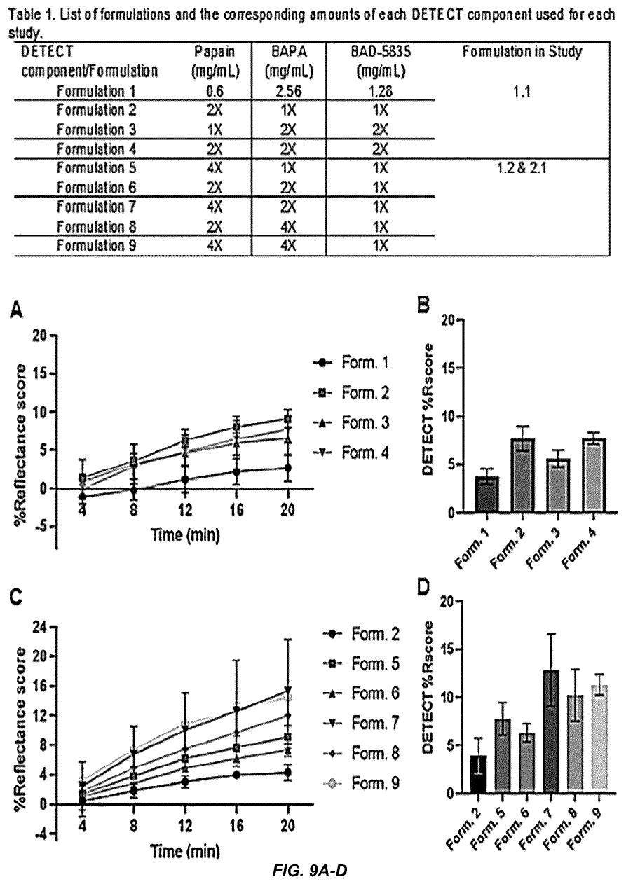

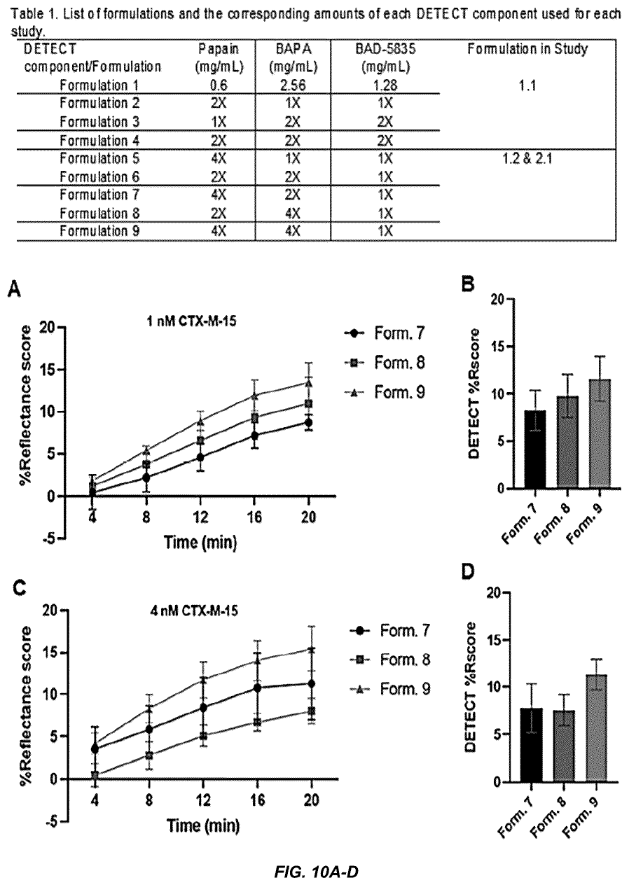

Study 2 Assessment of DETECT components on the test strip by CTX-M-15 activation using formulations 7-9. Given that formulations 7-9 gave the highest change in reflectance (% Rscore and DETECT % Rscore) in the cysteine activation study, these three formulations were used to assess the ability of the BAD-5835 β-lactamase small molecule targeting probe to detect the presence of recombinant CTX-M-15. If the small molecule targeting probe was not severely affected by the fabrication process, then in the presence of CTX-M-15, it will be hydrolyzed and liberate the triggering unit to initiate the signaling cascade thus providing the yellow colorimetric response. For this study, 1 nM or 4 nM of CTX-M-15 was chosen since these concentrations are above the dynamic range and undoubtedly will activate the DETECT system if the targeting probe did not undergo any modification during the fabrication process. For each formulation, two test strips were dedicated for an experiment run where one test strip received 20 μL of 1 nM or 4 nM of CTX-M-15 (sample) while the other test strip received 20 μL sodium phosphate buffer (control) and the percent reflectance were recorded (using 405 nm wavelength) every 4 minutes until 20 minutes of incubation time at ambient temperature had elapsed. The % reflectance score (% Rscore) and DETECT % Rscore were calculated for each formulation (see FIG. 10A-B) and obtained an % Rscore of-13 for formulation 9 which was strikingly like the response observed in the cysteine activation in study 1.2. Based on the DETECT % Rscore, each formulation provided modest responses in the presence of CTX-M-15 and there was a small difference between using more (formulation 9) or less (formulation 7) BAPA. The influencing factor for maximizing the change of reflectance continues to be the amount of papain that is used for fabricating the test strip. These results demonstrate that the DETECT components in the test strip format can detect the β-lactamase target biomarker CTX-M-15 and there was no indication the targeting probe suffered from the fabrication process. This shows the amplification system of DETECT in the test strip format is capable of detecting the presence of the β-lactamase CTX-M-15.

Highlights from Study 1 and Study 2:
(1) The β-lactamase targeting probe (BAD-5835) retained its ability to detect the presence of CTX-M-15.
(2) Increasing (2×→4×) BAPA provided a modest increase in the change of percent reflectance (% Rscore & DETECT % Rscore).
(3) Established order of coating reagents on reagent pad filter paper to furnish a working test strip for future studies (limit of detection, stability, material selection etc.).
(4) The reagent pad in this feasibility study-Ahlstrom-Munksjö Grade 222 filter paper.
(5) Drying temperature used (37° C.) and can be optimized to have a scalable fabrication process.
(6) Established at least 20 μL was useful amount of sample volume needed to prevent drying during incubation time.
(7) Papain activity was retained by demonstrating its activation by cysteine (Study 1.1 and 1.2). The targeting probe retained its ability to detect the presence of the recombinant CTX-M-15 β-lactamase (study 2).

Results of feasibility of translating assay components onto strip format by demonstrating activity of components is retained (relative to assay) by cysteine activation. Components can be successfully dried onto test strips and demonstrate levels of activity (by cysteine activation) comparable to other assay forms (20 min, ≤15% difference in activity). Additionally, BAD-5835 (G2B) β-lactam substrate did not show any sign of degradation that would result in false positive turn-on of system during fabrication.

It was found that papain was not affected by the fabrication process. Additionally, the control strip did not show any significant change in reflectance that would have otherwise resulted from papain activation by the liberated triggering unit that is released from degradation of the small molecule targeting probe (see FIG. 8A). Similarly, for study 2, two test strips were used to examine the ability of recombinant β-lactamase (CTX-M-15) to hydrolyze the small molecule targeting probe (BAD-5835) and trigger the amplification cascade. The sample test strip received 1 or 4 nM of β-lactamase, while the control received buffer and the reflectance was monitored every 4 minutes for 20 minutes (see FIG. 8B). There was significant change in reflectance which was indicative that the targeting probe was viable and recognizable by the β-lactamase. Although modest resolution between the two concentrations was observed, this was expected given that the two concentrations are well above the dynamic range for the CTX-M-15 target biomarker.

Improvements in the fabrication process of dry reagent test strips. Given that it was feasible to translate the DETECT components onto a dry reagent test strip format, there is significant room for improvement of other parameters such as the drying temperature, which will impact compatibility of the scale-up process and costs. To improve the fabrication process, optimization studies are conducted to evaluate the thickness, composition and pore sizes of membrane types, all of which influences the temperature for optimal drying time, and ability to absorb the materials that are being applied in the coating process. These optimization processes and determinations are well within the skill of one of skill the art and are routine. To develop a functional prototype of the U-DETECT test, the dry reagent test strip development will be approached in two phases.

Optimization of the liquid assay to dry reagents test strip conversion. The optimization work will begin by conducting a material (reagent pad matrix) screen with varying thickness, pore sizes and compositions. This screen will help identify materials with improved drying time, sample hydration ability (reagent pad must stay hydrated during reaction time) and change in signal with respect to background noise to retain sensitivity relative to the assay format. A minimum of two materials will be selected from this screen to move into drying condition optimization. Decreasing the drying time will greatly impact future scale up processes and help reduce manufacturing costs, therefore, it is equally important to identify an optimal temperature that will significantly reduce the drying time to less than 30 minutes. Lastly, minimizing the background noise that is generated will be further investigated by modifying the concentration of the targeting probe, e.g. BAD-5835, and modification of the buffer pH which can be a contributor to background noise (see FIG. 8).

Further optimization studies will be conducted with test strips that mimic the DETECT assay wells where the sample well contains one or more BAD probes, BAPA, and papain and control well only BAPA and papain. Further optimization studies will be performed with dBAPA that can provide faster time-to-results. One aspect will be ensuring that dBAPA does not get turned over during the fabrication step due to its reactivity with papain. Limit of detection studies will be performed to examine % decrease in sensitivity for recombinant target biomarkers. Further studies will be performed to determine the feasibility of swapping out the p-nitroaniline of BAPA with chromophore of higher wavelengths (525 nm or 625 nm).

Prototyping and verification of the U-DETECT test. Parameters established in the phase 1 optimization work will be used to develop a functional prototype test strip that includes a sample and control reagent pad in one test strip. In this prototyping phase, an accelerated thermal stability of test strips will be carried out at 2° C., 22° C., 37° C. and 45° C., where test strips will be assayed in triplicate once a week and compared to the baseline (2° C.—day 0). To determine the level of flexibility the U-DETECT test will have, sample application studies will be carried out. These studies will help determine whether dipping the test strip into the sample of interest, compared to sample addition by pipette will have an effect on analysis. To establish dipping as an application method, the dipping incubation time will need to be established, as well as removal of excess sample before instrument reading.

Once the parameters for a functional prototype have been established (i.e., dual reagent pad on a single test strip, information around stability of test strips, and information around sample application), a verification study with a panel of 96 bacterial clinical isolates, to ensure sensitivity and specificity is performed. A DETECT score that is greater than the threshold value will be considered positive, and the sensitivity and specificity of the modified assay will be determined by analyzing DETECT signals via a receiver operating characteristic (ROC) curve analysis. Accordingly, U-DETECT will offer improved outcomes for patients suffering from cUTI, by providing actionable information immediately to guide appropriate antibiotic treatment. Development of such a test will improve patient outcomes by informing early treatment decisions, enabling patients to receive the correct care sooner. This prevents disease progression to urosepsis, which carries mortality rates of 25-60%, and has a significant economic burden on healthcare systems. While the initial target condition is UTI, development of the U-DETECT strip test would allow for the rapid detection of ceftriaxone resistance in blood and from other sample types that are collected for diagnoses of various disease conditions. As such, market opportunities are far beyond the initial UTI market which can also extend to low- and middle-income countries (LMIC).

It will be understood that various modifications may be made without departing from the spirit and scope of this disclosure. Accordingly, other embodiments are within the scope of the following claims.

1. A solid substrate comprising reagents or components for measuring the presence of a β-lactamase produced by a Gram-negative, Gram-positive or pathogenic beta-lactam resistant bacteria, comprising:
at least a first pad on the solid substrate which comprises one, two or three components selected from the group consisting of (i) a targeting small molecule probe that is specificity acted on by a β-lactamase; (ii) a caged enzyme amplifier; and (iii) a chromophore-releasing small molecule indicator that is activated by an uncaged enzyme amplifier.

2. The solid substrate of claim 1, wherein the solid substrate comprises a second pad on the solid substrate which comprises the caged enzyme amplifier; and the chromophore-releasing small molecule indicator that is activated by the uncaged enzyme amplifier, wherein the second pad does not comprise the targeting small molecule probe;
optionally, a third or more pads on the solid substrate which comprises reagents or components.

3. (canceled)

4. The solid substrate of claim 2, wherein the first pad, the second pad, and the optional third or more pads comprise an adsorbent or plastic material.

5-9. (canceled)

10. The solid substrate of claim 1, wherein the targeting small molecule probe comprises a β-lactam group.

11. The solid substrate of claim 1, wherein when the targeting small molecule probe is acted upon by a β-lactamase the targeting small molecule probe releases a thiophenol group that interacts with the caged enzyme amplifier to uncage and activate the enzyme amplifier.

12. The solid substrate of claim 1, wherein the caged enzyme amplifier is a caged cysteine protease.

13. The solid substrate of claim 12, wherein the cysteine protease is selected from papain, bromelain, cathepsin K, calpain, caspase-1, separase, adenain, pyroglutamyl-peptidase I, sortase A, hepatitis C virus peptidase 2, sindbis virus-type nsP2 peptidase, dipeptidyl-peptidase VI, deSI-1 peptidase, TEV protease, amidophosphoribosyltransferase precursor, gamma-glutamyl hydrolase, hedgehog protein, and dmpA aminopeptidase.

14. The solid substrate of claim 1, wherein the caged enzyme amplifier is a caged papain enzyme.

15. (canceled)

16. The solid substrate of claim 1, wherein the chromophore-releasing small molecule indicator is a N-benzoyl-DL-arginine-4-nitroanilide (BAPA) or a derivative thereof.

17. The solid substrate of claim 16, wherein the derivative of BAPA comprises a different chromophore group for a p-nitroaniline group.

18. The solid substrate of claim 16, wherein the derivative of BAPA comprises a dipeptide or a tripeptide in place of the arginine group of BAPA.

19-20. (canceled)

21. The solid substrate of claim 1, wherein a chromophore is released from the chromophore-releasing small molecule indicator when acted on by the enzyme amplifier.

22. The solid substrate of claim 21, wherein the chromophore absorbs light in a wavelength from 350 nm to 900 nm.

23. The solid substrate of claim 22, wherein the chromophore is p-nitroaniline and absorbs light in a wavelength of about 405 nm.

24. (canceled)

25. The solid substrate of claim 22, wherein the chromophore is resorufin.

26. The solid substrate of claim 1, wherein the solid substrate comprises a third pad affixed onto the solid substrate which comprises the enzyme amplifier; and the chromophore-releasing small molecule indicator that is activated by the enzyme amplifier.

27. The solid substrate of claim 2, wherein the solid substrate comprises a fourth pad affixed onto the solid substrate which comprises reagents or components including the chromophore-releasing small molecule indicator that is activated by the enzyme amplifier.

28-54. (canceled)

55. A method to measure the presence of a β-lactamase produced by a pathogen in a sample, comprising:
contacting the solid substrate of claim 1 with the sample;
measuring light absorbance at a wavelength from 400 nm to 600 nm, wherein a measured change in light absorbance of the solid substrate is indicative that there is a β-lactamase produced by a pathogen in the sample.

56. The method of claim 55, wherein the β-lactamase is selected from TEM-1, SHV-1, CTX-M-14, CTX-M-15, CMY-2, and KPC-2.

57. The method of claim 55, wherein the pathogen is E. coli, K. pneumoniae, Pseudomonas aeruginosa and/or P. mirabilis.

58. (canceled)

59. The method of claim 55, wherein the sample is a urine or blood sample.

60. (canceled)

61. The solid substrate of claim 1, wherein the targeting small molecule probe has the general structure of Formula I: 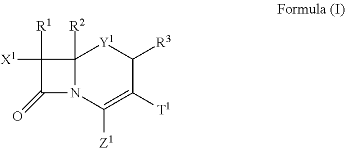
or a salt, stereoisomer, tautomer, polymorph, or solvate thereof,
wherein: T¹ is a benzenethiol containing group; Z¹ is a carboxylate, a carbonyl, or an ester;
X₁ is 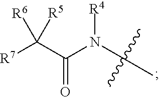
Y¹ is 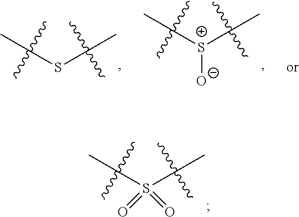
R¹-R⁶ are each independently selected from H, D, hydroxyl, nitrile, halo, amine, nitro, amide, thiol, aldehyde, carboxylic acid, alkoxy, optionally substituted (C₁-C₄) ester, optionally substituted (C₁-C₄) ketone, optionally substituted (C₁-C₆)alkyl, optionally substituted (C₁-C₆)alkenyl, optionally substituted (C₁-C₆)alkynyl, optionally substituted (C₅-C₇) cycloalkyl, optionally substituted aryl, optionally substituted benzyl, and optionally substituted heterocycle;
R⁷ is an optionally substituted (C₅-C₇) cycloalkyl, optionally substituted aryl, optionally substituted benzyl, or optionally substituted heterocycle.

62. The solid substrate of claim 61, wherein T¹ is a benzenethiol group selected from the group consisting of:

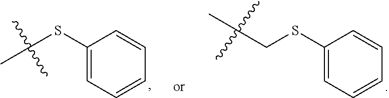 [SMILES: *Sc1ccccc1 ; *CSc1ccccc1]

63. The solid substrate of claim 61, wherein R⁷ is selected from the group consisting of:

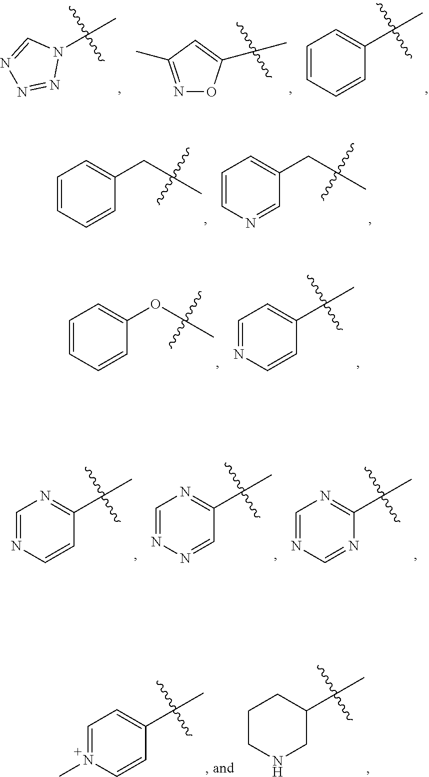

64-69. (canceled)

70. The solid substrate of claim 61, wherein the compound is selected from the group consisting of: 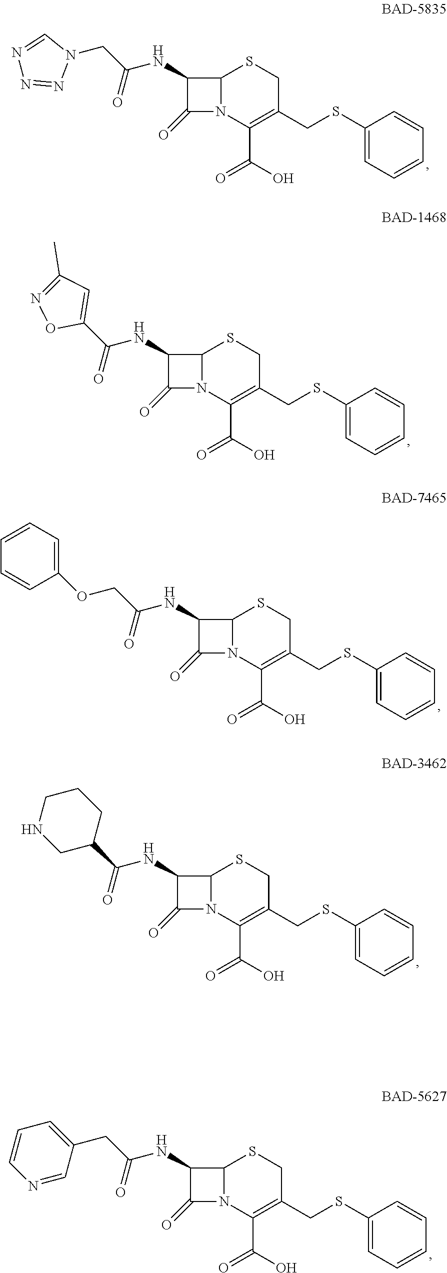
or a salt, stereoisomer, tautomer, polymorph, or solvate thereof.


SEQUENCE LISTING (1 sequence)

SEQ ID NO: 1 - protein, 6 aa, synthetic construct
QVVAGA